(19) United States
(12) Patent Application Publication
(10) Pub. No.: US 2016/0084814 A1
(43) Pub. Date: Mar. 24, 2016

(54) SUBMERSIBLE FLOW IMAGER

(71) Applicant: Woods Hole Oceanographic Institution, Woods Hole, MA (US)

(72) Inventors: Robert J. Olson, Cataumet, MA (US); Heidi M. Sosik, Falmouth, MA (US)

(21) Appl. No.: 14/961,158

(22) Filed: Dec. 7, 2015

Related U.S. Application Data
(63) Continuation-in-part of application No. 14/480,871, filed on Sep. 9, 2014.

(51) Int. Cl. G01N 33/18; G01N 15/14

(52) U.S. Cl. CPC G01N 33/1886; G01N 15/1404; G01N 2015/142

(57) ABSTRACT

Submersible technology capable of sampling and evaluating suspended particulate materials, including living and dead organisms, is disclosed herein. In addition to fluorescent imaging and analysis, the invention provides automated cell staining, cell sorting, particulate concentration, and organism recovery and preservation. These new technologies may be incorporated into a traditional “fully vertical flow path” cytometer configuration most suitable for anchored or suspended stationary embodiments, or in a novel modularized “low relief” configuration, suitable for integration into or on a submersible aquatic vehicle or in other low clearance installations, where it is important to limit the vertical height of the device.

### CROSS REFERENCE TO RELATED APPLICATIONS

This application claims priority to the provisional U.S. Patent Application No. 61/875,253 entitled “Submersible Flow Imager” filed Sep. 9, 2013 and the U.S. non-provisional patent application Ser. No. 14/480,871, entitled “Submersible Flow Imager,” and filed on Sep. 9, 2014. The entire specification, drawings, and claims of, now abandoned, U.S. patent application Ser. No. 11/978,246 (Publication No. U.S. 2009/0109432 A1), filed on Oct. 26, 2007, titled “Systems and Methods for Submersible Imaging Flow Apparatus” are completely incorporated in this application by reference. Additionally, the entire specification, drawings, and claims of U.S. Pat. No. 8,830,451 “Multinode Acoustic Focusing for Parallel Flow Cytometry Analysis Applications” filed May 9, 2011 are completely incorporated in this application by reference.

### STATEMENT REGARDING FEDERALLY SPONSORED RESEARCH OR DEVELOPMENT

Enhanced imaging flow cytometry via acoustic focusing and emulsion microfluidics is sponsored by National Science Foundation Grant No. OCE-1130140. Development of the Imaging FlowCytobot on autonomous vehicles is sponsored by National Science Foundation Grant No. OCE-1428703.

### REFERENCE TO A “SEQUENCE LISTING,” A TABLE, OR A COMPUTER PROGRAM

Not Applicable.

### DESCRIPTION OF THE DRAWINGS

The drawings constitute a part of this specification and include exemplary embodiments of the submersible flow imager, which may be embodied in various forms. It is to be understood that in some instances, various aspects of the invention may be shown exaggerated or enlarged to facilitate an understanding of the invention. Therefore, the drawings may not be to scale.

FIG. 1 shows an embodiment of the submersible flow imager when it is removed from its housing. Three plastic standoffs prevent contact of the components and housing during installation. The left image is a front view, showing the “fluidics and optics” side of the optical systems. The flow cell (hidden by standoff) is located between the condenser and objective lenses. The right image is a side view and the optical system is edge-on in the center with the fluidics/optics components mounted to the left and the electronics to the right. FIG. 1 depicts the submersible flow imager in a substantially vertical orientation. For operation in a substantially horizontal orientation, the location of the components would be the same except rotated approximately 90 degrees clockwise (or counter-clockwise).

FIG. 2 is a depiction of acoustic focusing elements of one embodiment of the submersible flow imager. The drive transducer on the focusing tube sets up the acoustic standing wave, and the pick-up transducer monitors its frequency for use in feedback regulation. The focused particles at the end of the tube enter the submersible flow imager's existing flow and detection system.

FIG. 3 depicts the modules in one embodiment of the submersible flow imager.

FIG. 4 depicts an analysis of uniform fluorescent beads which illustrates measurements of fluorescence and scattering.

FIG. 5 depicts flow cytometric measurements of side scattering and chlorophyll fluorescence, and selected images of phytoplankton cells in a seawater sample from Woods Hole Harbor, which were analyzed using the imaging apparatus (triggered by chlorophyll fluorescence).

FIG. 6 is a chart depicting the process for microfluidic injection of sorted cells into storage tubes.

FIG. 7 is a diagram of the flow path of one embodiment of the submersible flow imager.

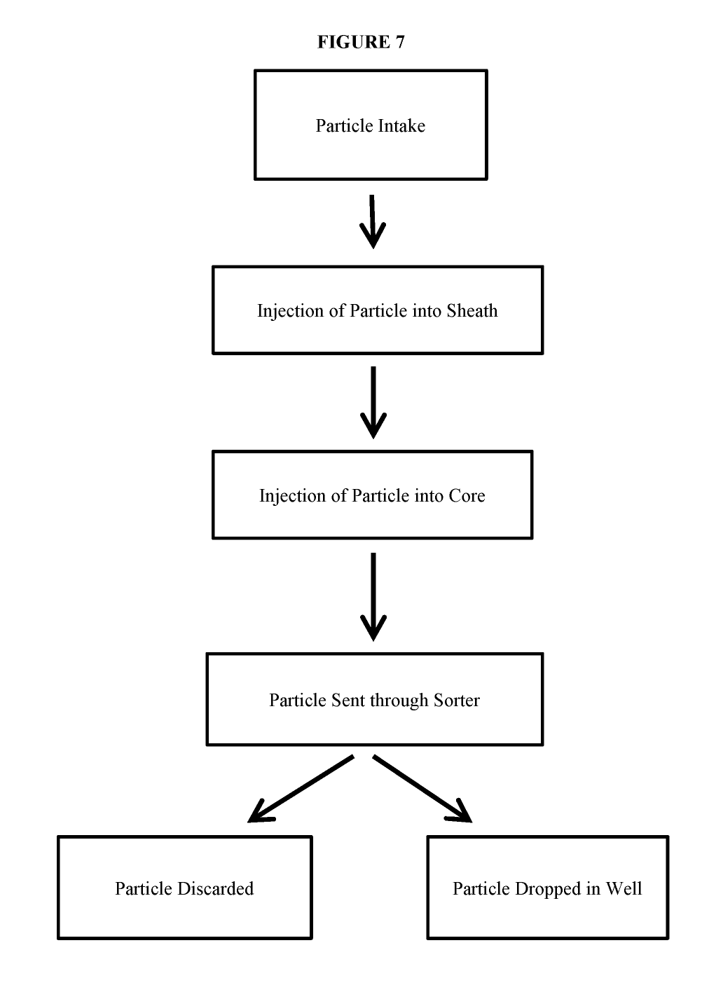

FIG. 8 is depicts embodiments of the invention where a catcher tube is employed as part of the sorting system.

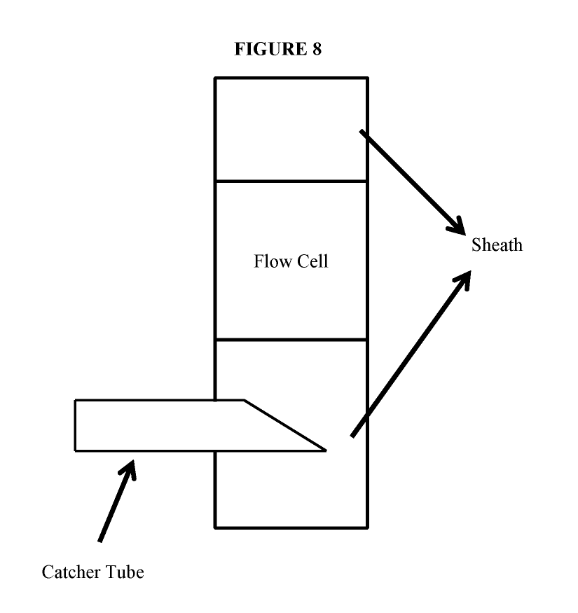

FIG. 9 schematically depicts an embodiment of the fluidics for IFCB-S.

FIG. 10 depicts embodiments of optical layouts for IFCB-S in staining mode (left panel) and non-staining mode (right panel).

FIG. 11 depicts examples of ciliates at MVCO as imaged by one embodiment of the standard IFCB triggering on chlorophyll fluorescence.

FIG. 12 depicts examples of dinoflagellates at the WHOI dock as imaged by IFCB-S triggering on FDA and chlorophyll fluorescence (boxes A and B) and actively grazing dinoflagellates sample at MVCO as imaged by a standard IFCB triggering on chlorophyll fluorescence (box C). (A) contains dinoflagellates with low chlorophyll and high stain fluorescence; (B) contains dinoflagellates with both high chlorophyll and stain fluorescence.

FIG. 13 depicts FDA fluorescence versus side angle light scattering (peak signals) for a scuticociliate culture. (A) Non-stained cell culture triggering on side scattering; (B) Non-stained cell culture triggering on stain fluorescence; (C) Stained cell culture triggering on stain fluorescence. Black symbols indicate detrital particles and gray points are scuticociliates, as determined from visual inspection of associated images.

FIG. 14 depicts a comparison of flow cytometric detection of a scuticociliate culture stained with FDA or LTG. Solid line is best fid. Dashed line is 1:1. 95% confidence intervals are shown for each count. Correlation with 1:1 line is 0.99.

FIG. 15 depicts cell concentrations (cells ml⁻¹) for ciliate mix, Mesodinium sp., Laboea Strobila, tintinnids, Gyrodinium sp., and Protoperidinium sp. Comparing results from manual microscopy with samples analyzed by IFCB-S operated in staining and non-staining modes. Samples were collected from Woods Hole Harbor in winter (A—Jan. 19, 2014), spring (B—May 11, 2014), summer (C—Jul. 2, 2014), and fall (D—Oct. 18, 2014), with manual microscopy only available for winter and fall. Error bars indicate 95% confidence intervals computed assuming Poisson distributed counting statistics. Significance is indicated using dots; black and gray dots are significantly different from each other. If no significant differences were found within a taxonomic group, no bars are displayed.

FIG. 16 depicts daily-binned cell concentration for total ciliates and gyrodinoid dinoflagellates imaged on Aug. 25, 2014 during the ECOMON cruise. Light and dark grey bars indicate populations with high and low red fluorescence, respectively. Error bars indicate 95% confidence intervals computed assuming Poisson distributed counting statistics.

FIG. 17 depicts (A) regression between hourly bins of manually identified Laboea strobila cell abundances at MVCO and automated classification results for score threshold 0.7 The black line represents a 1:1 line and the gray line is best fit; (B) r² values for all thresholds tested; (C) y-intercept values of best fit line for all thresholds tested; (D) slope values of best fit line for all thresholds tested. Vertical gray line in B-D indicates selected threshold score of 0.7.

FIG. 18 depicts daily resolution time series of Laboea strobila cell abundance at MVCO. Intermittent (approximately 2-week interval) counts from manual identification (small dots) are shown with the high-resolution results from automated classification (black line).

FIG. 19 depicts an embodiment of the sorting subsystem of the IFCB-Sorter.

FIG. 20 depicts an embodiment of the IFCB-Sorter sorting subsystem signals and controls. (A) depicts the initial trigger seen by the IFCB; (B) depicts the hold pulse to inhibit triggers until sorting is complete; (C) depicts the catcher tube deployment pulse; (D) depicts the sorting subsystem PMT trigger; (E) depicts the upstream solenoid closure; (F) depicts the downstream solenoid closure; (G) depicts the microinjector pulse; and (H) depicts the autoclone pulse to advance well plate.

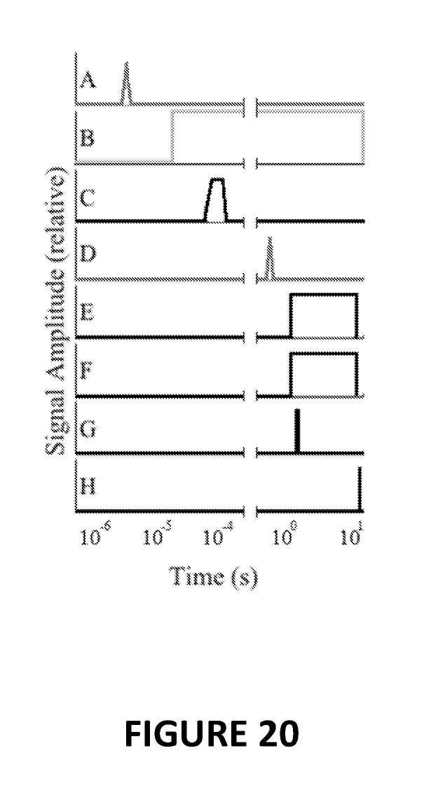

FIG. 21 depicts (A) the capture efficiency with varying catcher tube deployment pulses and (B) the capture efficiency with differing deployment delays and total time held at 900 μs.

FIG. 22 shows orientation and motion only had minor effects on the position of the imaged particles across the flow cell channel. Dots indicate positions of cell images during 16 minutes of analysis of a mixture of large and small cultured phytoplankton. Regions with no particles on this plot correspond to the particle-free sheath fluid that surrounds the seawater sample core. IFCB was tilted from vertical to horizontal, rocked by 30 degrees with a period of approximately 5 seconds, and returned to vertical, as indicated by the top labels. Images remained well-focused even while the instrument was horizontal and rocked to simulate wave motion.

FIG. 23 depicts one illustrated embodiment of the SV2 Wave Glider with a subsea payload area at the deepest point for the integration of the Low Relief IFCB.

FIG. 24 shows one illustrated embodiment of one low relief configuration of the specific orientation (i.e., flow cell) and/or proximity of the modules.

### DETAILED DESCRIPTION

Described herein, in various embodiments, are systems, devices, and methods for imaging, staining, sorting, and/or collecting microorganisms. In one embodiment, the system comprises a low relief, device for processing particles within liquid suspensions. In some embodiments, the device is modular. The system comprises a flow system, a detection system adapted to detect one or more aspects of the suspended particles passing through the detection system, an electronics system electronically connected to said flow system and said detection system, and a watertight housing further comprising the flow system, the detection system, and the electronics system. The liquid suspension is imbibed into the flow system and transferred to the detection system for particle processing. In other embodiments, the device may further comprise a bubble minimization assembly capable of removing bubbles from the flow system, particularly when the device is substantially horizontal. However, the bubble minimization assembly may be present in embodiments where the device is not substantially horizontal. The bubble minimization assembly functions to remove bubbles and comprises one or more tubes and a valve for the syphoning and expulsion of the bubble-containing fluid to the outside environment. In some embodiments, the flow system comprises a sample intake means configured to imbibe a liquid suspension from the surrounding environment, a pumping means providing motive force of the imbibed liquid, a delivery means capable of transferring the liquid suspension from the sample intake means to a detection system, and at least one valve. In some embodiments, the detection system comprises a detection path coupled to the pumping means through a delivery means, a detection interface subsystem further comprising a flow cell, a hydrodynamically focused core, and a sheath adapted for the flow of a sheath fluid, and an optical system. In some embodiments, the system comprises an acoustic focusing element which performs the function of in-line concentration of flowing samples in the flow system. In some embodiments, the electronics system comprises a controller, a data acquisition device, a power source, and a data processor and said electronics system provides control for movement of a liquid suspension within a flow path, the detection of particles, and data storage and transmission. In some embodiments, the system comprises a particle suspension means, the particle suspension means comprising at least one of the following: a syringe pump to impart turbulence, a mixing device, a magnetic stirring bar, a flow path having a cork-screw shape, wave action of the body of water in which the device is located, or a combination thereof.

In one embodiment, the device comprises a low relief system. In some embodiments, the device comprises a low relief profile and its height is dictated by the height of the detection path. In a further embodiment, the device comprises a low relief profile and the height of the device is less than 48 cm. Is still a further embodiment, the device comprises a low relief profile and the height of the device is less than 36 cm. In some embodiments, the device comprises a low relief profile and the device further comprises an attachment means for mounting on a vehicle.

In another embodiment, the device comprises an anti-fouling system to reduce bio-fouling of the flow system and the detection system.

In another embodiment, the device performs particle processing including, but not limited to, at least one of the following: imaging, staining, sorting, observing, counting, isolating, or any combination thereof.

In another embodiment, the device comprises an automated staining system and the automated staining system comprises a microinjector, a stain to dye a particle for imaging, a stain reservoir to contain a stain, and a mixing chamber capable of receiving a stain from the stain reservoir via a delivery means. The sample intake means intakes a sample, which comprises particles, and the microinjector adds a stain from the stain reservoir to the mixing chamber. The pumping means pushes the sample into said mixing chamber, where the sample mixes and incubates with the stain; then the pumping means pulls the sample back through the delivery means and into the sample syringe, and the flow system delivers the sample into the flow cell for analysis. The stain is selected from the group comprising: membrane-permeant calcein AM, membrane-impermeant ethidium homodimer-1, DiOC18(3), SYBR® 14 nucleic acid stain, membrane-impermeant propidium iodide, C12-resazurin, SYTOX® Green dye, Hoechst nucleic acid stain, fluorescein diacetate (FDA), or any combination thereof. Additionally, the stain could include any stain that is either now known in the art or that is discovered in the future, which accomplishes a function similar to the stains listed above.

In another embodiment, the device comprises an automated sorting system and the automated sorting system comprises a catcher tube positioned to receive one or more particles from the flow cell, a piezo element adapted to position the catcher tube to receive a particle from the flow cell, a valve capable of shutting off flow of the flow cell to allow the particle to be analyzed, and a laser and detector assembly. The laser and detector assembly function to verify that a sample particle was actually captured and to enable the correctly timed release of a drop the sheath fluid containing the sample particle to be preserved.

In another embodiment, the system comprises a vehicle and a low relief device which is capable of processing particles within liquid suspensions. In this embodiment, the system comprises a vehicle, that is adapted to receive a low relief device within the vehicle's hull and the low relief device is capable of use in water, a flow system, a detection system adapted to detect one or more aspects of the suspended particles passing through the detection system, an electronics system electronically connected to said flow system and said detection system, and a watertight housing; wherein the flow system, the detection system, and the electronics system are disposed within the housing. In some embodiments, the housing of the low relief device is removably attached to the vehicle. In some embodiments, the low relief device, that is removably attached to the vehicle further comprise a bubble minimization assembly which functions to remove bubbles from the flow system particularly when the device is substantially horizontal. However, in other embodiments, the bubble minimization assembly functions to remove bubbles from the flow system when the device is not substantially horizontal. In one embodiment, the bubble minimization assembly functions to remove bubbles and comprises one or more tubes and a valve for the syphoning and expulsion of the bubble-containing fluid to the outside environment. In some embodiments, the flow system comprises a sample intake means configured to imbibe a liquid suspension from the surrounding environment, a pumping means providing motive force of the imbibed liquid, a delivery means capable of transferring the liquid suspension from the sample intake means to a detection system, and at least one valve. In some embodiments, the detection system comprises a detection path coupled to the pumping means through a delivery means, a detection interface subsystem further comprising a flow cell, a hydrodynamically focused core, a sheath adapted for the flow of a sheath fluid, and an optical system. In some embodiments, the system comprises an acoustic focusing element which performs in-line concentration of flowing samples in the flow system. In some embodiments, the electronics system comprises a controller, a data acquisition device, a power source, and a data processor; the electronics system provides control for movement of a liquid suspension within said flow path, the detection of said particles, and data storage and transmission.

In some embodiments, the device comprises a low relief profile to mount onto a vehicle and the height of the device is dictated by the height of the detection path of the device. In a further embodiment, the device has a height of less than 48 cm. In another embodiment, the device has a height of less than 36 cm. In still another embodiment, the device has a height of less than 20 cm.

In another embodiment, the system comprises a vehicle and a device, where the device is removably included within the hull of the vehicle. The device comprises an anti-fouling system to reduce bio-fouling of the flow system and the detection system.

In another embodiment the device comprises an automated staining system. The automated staining system comprises a microinjector, a stain to dye a particle for imaging, a stain reservoir to contain a stain, and a mixing chamber capable of receiving a stain from the stain reservoir via a delivery means. The sample intake means intakes a sample, which comprises particles, and the microinjector adds a stain from the stain reservoir to the mixing chamber. The pumping means pushes the sample into said mixing chamber, where the sample mixes and incubates with the stain; then the pumping means pulls the sample back through the delivery means and into the sample syringe, and said flow system delivers the sample into the flow cell for analysis. The stain is selected from the group comprising: membrane-permeant calcein AM, membrane-impermeant ethidium homodimer-1, DiOC18(3), SYBR® 14 nucleic acid stain, membrane-impermeant propidium iodide, C12-resazurin, SYTOX® Green dye, Hoechst nucleic acid stain, fluorescein diacetate (FDA), or a combination thereof. Additionally, the stain could include any stain, which is either now known in the art or that is discovered in the future, which accomplishes a function similar to the stains listed above.

In another embodiment, the device comprises an automated sorting system; the automated sorting system comprises a catcher tube positioned to receive one or more particles from the flow cell, a piezo element adapted to position the catcher tube to receive a particle from the flow cell, a valve capable of shutting off flow of the flow cell to allow the particle to be analyzed, and a second laser and detector assembly to verify that a sample particle was actually captured and to enable the correctly timed release of a drop the sheath fluid containing the sample particle to be preserved.

In another embodiment, the system comprises providing a vehicle and a low relief device; the low relief device is capable of being loaded into the hull of the vehicle. In a further embodiment, the low relief device performs particle processing, which includes, but is not limited to, imaging, staining, sorting, observing, counting, isolating, or a combination thereof.

The systems, apparatus, and methods described herein relate to a submersible flow imaging apparatus that can sample, monitor, evaluate, sort, stain, and/or preserve individual microorganisms in aquatic environments by imbibing liquid samples and processing them according to the features described herein.

The system, apparatus, and methods of the submersible flow imager are described with specificity herein to meet statutory requirements. However, the description itself is not intended to necessarily limit the scope of claims. Rather, the claimed subject matter might be embodied in other ways to include different steps or combinations of steps similar to the ones described in this document, in conjunction with other present or future technologies. Although the terms “step” and/or “block” or “module” etc. might be used herein to connote different components of methods or systems employed, the terms should not be interpreted as implying any particular order among or between various steps herein disclosed unless and except when the order of individual steps is explicitly described.

Furthermore, the described features, structures, or characteristics may be combined in any suitable manner in one or more embodiments. One skilled in the relevant art will recognize that the submersible flow imager and its systems and methods may be practiced without one or more of the specific details, or with other methods, components, materials, and so forth. In other instances, well-known structures, materials, or operations are not shown or described in detail to avoid obscuring aspects of the invention.

In the face of aquatic pollution, climate change, and other environmental challenges which impact aquatic life, the ability to evaluate the status of the microscopic aquatic biome has significant commercial importance. Plankton in the size range of 10-100 micron, including many diatoms and dinoflagellates, are one critical component of aquatic ecosystems, but due to the difficulties with sampling in dynamic aquatic environments, many aspects of their status and distribution are poorly understood.

In the past, submersible flow cytometers were used to measure fluorescence and light scattering signals from a laser beam to characterize the smallest phytoplankton cells (approximately 1-10 μm). Other commercially available instruments, such as the Video Plankton Recorder are capable of monitoring plankton at the other end of the size spectrum (mainly zooplankton>approximately 100 μm). However, plankton in the size range of approximately 10-100 μm are not well sampled by either of these instruments. This is a critical gap because phytoplankton in this size range, which includes many diatoms and dinoflagellates, can be especially important in coastal blooms, while microzooplankton, such as protozoa, are critical to the diets of many grazers including copepods and larval fish.

Other submersible flow cytometers have been developed, such as the FlowCAM and the CytoSub, but none has the necessary resolution and field endurance for many of the needed ecological studies. The FlowCAM differs from the submersible flow imager disclosed herein (marketed under the name “Imaging FlowCytobot” and also referred to herein as “IFCB”) in that the FlowCAM does not utilize hydrodynamic focusing of the sample flow inside a particle-free sheath fluid; the FlowCAM is therefore subject to flow cell fouling by particles adhering to the flow cell walls. Lack of the hydrodynamic focusing of sample also means that many images taken by FlowCAM are out of optical focus. The submersible flow imager described herein differs from the CytoSub in that the IFCB disclosed herein is primarily an imaging instrument and captures a high-quality image of every triggering particle, while the CytoSub uses imaging only for identification of user-specified regions of light scattering and fluorescence signatures. In addition, images from the CytoSub are of lower quality than those from the IFCB, as indicated by the Alexandrium (A. Sanguina) image on the CytoSub website (http://www.cytobuoy.com/products/benchtop/) accompanied by the statements that its “tail [i.e., flagellum] cannot be seen on the picture, but is present in the pulse shape!” Dinoflagellate flagella are routinely observed in the IFCB images. Neither the FlowCAM nor the CytoSub has demonstrated the capability for continuous operation in the field over 6 months duration deployments, as has the IFCB.

As used herein, references to the words “vertical” or “vertically oriented” mean that the element discussed is substantially aligned with the direction of gravity. For example, if the term “vertically oriented” is used to describe the flow path, or other pathway, it means that the flow path or other pathway is substantially positioned such that the particles flow through the pathway in the direction of the gravitational pull of the earth on such particles. The term “non-vertical orientation,” as used herein, includes any orientation that is not “substantially vertical,” including, but not limited to, orientations that are substantially horizontal.

As used herein, the terms “particles” or “particulate matter” includes within its definition cells and organisms, even if a specific reference to cells and/or organisms is not made.

According to various aspects, the disclosure herein relates to an apparatus, systems, and associated methods for imaging, sorting, and/or staining particles within liquid suspensions. The submersible flow imager comprises a flow system, a detection system, and an electronics system. The flow system is the means by which the sample particles are imbibed into the submersible flow imager, transported through the different modules or other systems of the submersible flow imager, and then either stored or discarded from the submersible flow imager. The electronics system provides control both for the flow system, in controlling the movement of the suspensions within the flow path, and the detection system, in controlling the detection of the particles within the suspension. The electronics system also provides for data storage and data transmission.

The submersible flow imager comprises a flow system, a detection system, an electronics system, and a housing. Each of these systems is described below. Specific embodiments of the submersible flow imager may employ multiple versions of any particular system (flow system, detection system, and/or electronics system) described herein depending on the particular application of the submersible flow imager. The housing may comprise any material known in the art to be suitable for the conditions in which the submersible flow imager will be deployed; such materials include metals, plastics, polymers, ceramics, etc. In one embodiment, the housing is fabricated from aluminum. In another embodiment, the housing is waterproof to protect the components disposed within. In one embodiment, the submersible flow imager further comprises a mounting interface for mounting the submersible flow imager onto a vessel, vehicle, or platform. The mounting interface is the means by which the attaching means is attached to the housing. The mounting interface comprises any interface known in the art for use in attaching items to each other, including attached metal loops and having the housing formed such that a cutout in the housing can have a cable/rope passed through it to secure the housing to another object. In another embodiment, the submersible flow imager housing comprises an attachment means for attaching the submersible flow imager to a vessel, vehicle, or platform for towing. In one embodiment, the attachment means provides a means of removably attaching the submersible flow imager to a vessel, vehicle, or platform. In another embodiment, the attachment means provides a means for permanently attaching the submersible flow imager to a vessel, vehicle, or platform. The attachment means can be any attachment means known in the art, such as hook and latch, metal or plastic fasteners, ropes, cable, welding, etc. In one embodiment, the submersible flow imager is operational (e.g., imbibing sample particles) while being towed. In another embodiment, the submersible imager is operational while being in a substantially stationary position. In another embodiment, the submersible flow imager is operational while being located in strong wave action areas.

Operation of the submersible flow imager in the water in a towed arrangement (either while being towed or while stationary) simplifies temperature control and elimination of particles and sheath fluid. Additionally, operation of the submersible flow imager under water results in the submersible flow imager being exposed to less motion from wave action or other causes, thus enhancing the operation of the submersible flow imager. Further, operation of the submersible flow imager under water allows samples to be taken at different depths. In one embodiment, the depth at which samples are imbibed is controlled through the adjustment of the towing angle. In a further embodiment, the submersible flow imager comprises a pressure (depth) sensor to detect the depth at which samples are imbibed. In another embodiment, the depth at which samples are imbibed comprises adjusting the speed of the vehicle, vessel, or platform while the sample is being imbibed.

In another embodiment, the submersible flow imager is deployed in the hull of a vessel, vehicle, or platform. In one embodiment, the flow cell remains substantially vertical while the other aspects of the submersible flow imager are substantially horizontal. In another embodiment, the flow cell remains substantially vertical while the other aspects of the submersible flow imager are positioned in a non-vertical orientation. In another embodiment, the detection system remains substantially vertical while the other aspects of the submersible flow imager are positioned in a non-vertical orientation. In another embodiment, all aspects of the submersible flow imager are positioned in a non-vertical orientation. In yet another embodiment, all aspects of the submersible flow imager are substantially horizontal.

In one embodiment, the submersible flow imager is modularized (e.g., organized into separate systems or components). In this embodiment, at least one module is located in a separate housing than the other modules. In this embodiment, the modules comprise the flow system, the optical system, and the electronics system. In one embodiment, the flow system module and the optical system module are in one housing while the electronic system module is in a separate housing. In another embodiment, all three modules are each contained it their own separate housings. In a further embodiment, the housing where the flow system and optical system modules are located is comprised by the payload bay area of the vessel, vehicle, or platform. In yet another embodiment, the electronics system module is comprised by the electronics system of a vessel, vehicle, or platform and the flow system module and optical system module are located in separate housings from one another. In another embodiment, the optical system module housing is comprised by the payload bay area of a vessel, vehicle, or platform.

Particles to be Analyzed.

The submersible flow imager is used to obtain and evaluate liquid samples containing suspended particulate matter including inanimate as well as living and/or dead cells/organisms. In some embodiments, the particles are autofluorescent or can be stained with dyes. In one embodiment, the dyes are fluorescent dyes. In other embodiments, the particles are detected by light scattering. In still other embodiments, the particles comprise another easily detected property, such as conductivity or opacity. In yet a further embodiment, the particles are autofluorescent, or can be stained with fluorescent dyes, and the particles are detected by light scattering and/or conductivity or opacity.

Cell-Based Particles.

In some embodiments, the particles to be detected are living cells/organisms which are autofluorescent. In the case of the evaluation of cells/organisms without autofluorescence, or previously living, but now-dead cells/organisms, specific dyes, dye systems, or chemical stains, which are known in the art to stain such cells/organisms, may be useful. In other embodiments, light scattering methods are used to detect cell/organisms. In yet a further embodiment, the particles to be detected are autofluorescent or can be stained with fluorescent or other suitable dyes, which are either known in the art or which may be discovered in the future, and the particles are detected by light scattering and/or conductivity or opacity.

Particle sizes capable of being analyzed by the submersible flow imager will be dictated, at least in part, by the diameter of the core, which comprises the imbibed sample, which flows through the sheath tubing, and the flow cell. The core is surrounded by the sheath fluid while flowing through the sheath. In general, particles will have a smaller (in the shortest dimension) cross-sectional diameter than the cross-sectional diameter of the flow cell. Thus for a cross-sectional diameter of approximately 180 μm, particles of approximately 150 μm or less may be analyzed. Larger particles may be analyzed with larger flow cells (e.g., approximately 360 micron flow diameter enables analysis of cells greater than approximately 150 μm, preferably greater than approximately 200, 250, or 300 μm).

Other suspended particulates, such as detritus, empty diatom frustules, and heterotrophic organisms may also be evaluated in the submersible flow imager.

In one embodiment, the submersible flow imager comprises a flow system, a detection system, an electronics system, an anti-fouling means, and a housing. The flow system is the system by which the particles to be detected are imbibed by the submersible flow imager and then transported through the submersible flow imager, including through the detection path of the detection system. The detection system is the system which detects certain aspects of the sample particles. The electronics system regulates the flow of sample particles through the flow system, the operation of the detection system, and the operation of the anti-fouling means. The anti-fouling system attempts to prevent and/or remove the bio-fouling of elements of the submersible flow imager that are subject to bio-fouling.

In some embodiments, the sample particles are manipulated and/or analyzed by particle processing which includes, but is not limited to, imaging, staining, sorting, observing, counting, isolating, or a combination thereof.

The Housing.

The housing is appropriately waterproof (e.g., watertight, pressure-tight) and provides a protective structure around the flow system, the detection system, the electronics system, and the anti-fouling system. In another embodiment, the submersible flow imager comprises multiple housings, with modules of the submersible flow imager being located in separate housings. In another embodiment, the electronics system is located in one housing, and the remaining systems are located in a separate housing. In yet another embodiment, the housing of the submersible flow imager is the payload bay of a vehicle. In yet another embodiment, the vehicle is an unmanned vehicle, such as a glider, an autonomous underwater vehicle (AUV), a remotely operated vehicle (ROV), a hybrid remotely operated vehicle (hROV), an autonomous kayak or surface vehicle, or the like. In one embodiment, the glider is a Wave Glider 3. In one embodiment, the surface vehicle is a Jet-Yak.

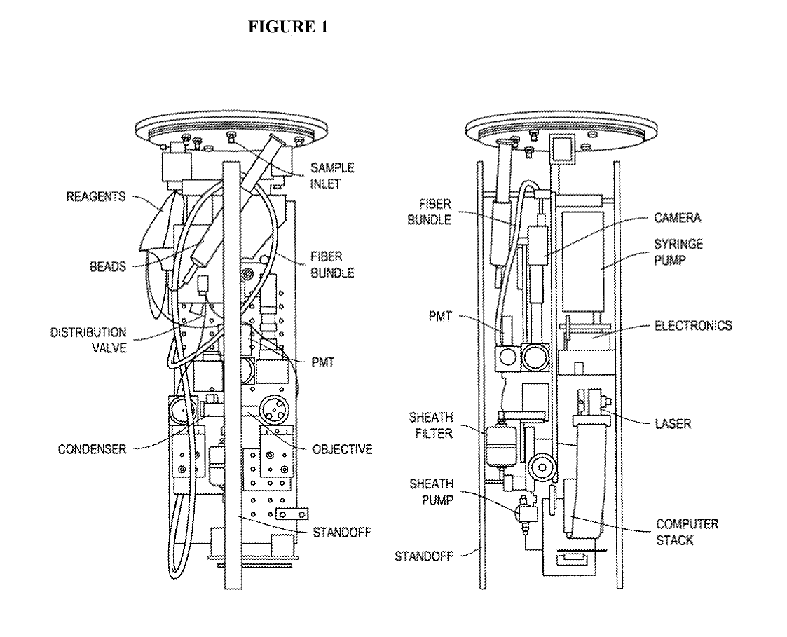

In an embodiment, the imaging apparatus is constructed around an optical breadboard (e.g., with dimensions of 20.32×60.96 cm) comprised of one or more off-the-shelf components. The fluid-handling and electronics components are mounted on opposite sides of the breadboard (FIG. 1). The breadboard hangs from the end cap of the submersible flow imager. The end cap, when secured in place, creates a seal with the housing of the submersible flow imager. In one embodiment, the end cap creates a watertight seal with the housing through the use of two nitrile o-rings or other suitable seal. In one embodiment, the seal is made of any material, whether now known or discovered in the future, which is capable of providing a water-tight seal. Non-limiting examples of seals include, gaskets, fittings, and sealants. In one embodiment, the end cap comprises connections for transmitting data. In another embodiment, the end cap comprises external connections to an observatory guest port for power and Ethernet communication (e.g., fiber optic) with the shore or devices capable of receiving such communications, regardless of whether they are located on shore. In one embodiment, the external connections comprise fiber optics. In another embodiment, the external connections comprise any method, device, or signal that is now known or discovered in the future that is capable of sending communications from the device to the shore or other devices capable of receiving such communications, regardless of whether they are located on shore. In yet another embodiment, communication between the electronics system and the connections for transmitting data is effected via cable. In a further embodiment, the communication between the observatory guest port and the electronics system is at least approximately 1 megabit/second. In another embodiment, the communication between the observatory guest port and the electronics system is approximately 10 megabits/second. In still another embodiment, the communication between the observatory guest port and the electronics system is at least approximately 10 megabits/second or higher. In another embodiment, the cable is a Category-5 cable, Category-5e cable, or other suitable means of communication transmission as known in the art. In still another embodiment, communication between the submersible flow imager and the shore or devices capable of receiving such communications, regardless of whether they are located on shore, is accomplished using optic fiber.

In one embodiment, the electrical system comprises a power supply. In another embodiment, the power supply comprises 36 V DC (100 W).

The Flow System.

The flow system comprises the flow path. The sampled liquid suspension, which comprises particles to be analyzed, travels within the submersible flow imager device, from imbibition through to either disposal or preservation. Any suitable means known in the art may be employed as a flow path (e.g., a tube, hose, or other hollow object that is capable of transporting liquid). In one embodiment, the flow path comprises a tube or hose. The flow path comprises the sheath tubing and the sheath fluid. In many embodiments, all or part of the flow path is comprised of materials resistant to fouling. Typical fouling-resistant materials include, copper, Teflon, and other hydrophobic materials. In other embodiments, the flow path is comprised of materials stable to treatment with the preferred anti-fouling agents such as sodium hypochlorite and surfactants. The flow system further comprises systems for recycling imbibed fluids and the sheath fluid. In some embodiments, functionally, the flow system often comprises 7 specific subsystems. The subsystems comprise: (i) a sample intake means, (ii) a pumping means, (iii) a particle suspension means, (iv) a delivery means, (v) a detection path, (vi) a detection interface, and (vii) a valve control interface. Each subsystem may be present singly or multiply depending on the needs of the specific embodiment.

In one embodiment, the flow system of the apparatus is based on that of a conventional flow cytometer—hydrodynamic focusing of a core, which comprises the sample stream, into the sheath fluid carries the particles to be analyzed in a single file fashion through a laser beam and then through the optical system's field of view. In another embodiment, the flow system is substantially horizontal. In another embodiment, the flow system is substantially vertical. In still another embodiment, part of the flow system is substantially vertical while other parts of the flow system are positioned in a non-vertical orientation.

In one embodiment, the flow system comprises a substantially vertical detection path and a detector interface. In another embodiment, the flow system comprises a substantially horizontal detection path and a detector interface. In one embodiment, portions of the flow path are substantially vertical while other portions of the flow path are in a non-vertical orientation. In another embodiment, portions of the flow path are substantially vertical while other portions of the flow path are substantially horizontal. In accordance with one embodiment of the flow system, the flow system further comprises a non-vertical delivery means. In an embodiment, the submersible flow imager is substantially horizontal while being towed and substantially vertical while at rest. In a further embodiment, whether the submersible flow imager is substantially horizontal or substantially vertical depends on the speed at which the submersible imager is travelling, either by being towed or while mounted to a vessel, vehicle, or platform. Additionally, the electronics system may be substantially, spatially removed from the flow system. In accordance with other embodiments, the device also comprises at least one of the following: a particle staining system, a particle sorting system, and/or a particle concentrating system.

In one embodiment, a sample is injected into the center of a sheath within the flow path. In some embodiments, the sheath fluid is injected into the flow system in an even stream using at least one jet and in some embodiments two or more jets. The sample can be any appropriately sized particles in suspension, including, but not limited to, blood, sediment in water, lake water, fresh water, marsh water, and seawater. In an embodiment, the sample comprises seawater. The sheath tubing can be made of any material that is known in the art, such as plastic, polymers, metal, etc. The sheath comprises a flow of sheath fluid, which is particle-free. The sheath fluid comprises a liquid. The liquid for the sheath fluid can be water, a surfactant, an antimicrobial additive, a buffered solution, a salt solution, or any other fluid or liquid that is capable of carrying particles through the flow system, whether now known or discovered in the future. In one embodiment, the sheath fluid is water. In another embodiment, the sheath fluid comprises a liquid comprising a surfactant. In another embodiment, the sheath fluid comprises a liquid comprising an anti-microbial additive. In still another embodiment, the sheath fluid comprises a buffered solution. In yet another embodiment, the sheath fluid comprises a salt solution. In other embodiments, the sheath fluid comprises a liquid that is capable of carrying particles through the flow system. The injection of the sample into the sheath results in the sample core being confined to the center of the flow of the sheath fluid, with the sheath fluid substantially creating a “collar” around the sample. Surrounding the sample with the sheath fluid results in the sample, and thus the particles to be analyzed, being substantially confined to the center of the flow cell in the detection system, and helps ensure that the particles to be analyzed are in focus as they pass through the optical system, which is part of the detection system. In one embodiment, the sheath fluid is recycled through a filter cartridge, which removes particles after they have been analyzed by the detection system. In another embodiment, the sheath fluid is disposed into a waste container located within the flow system. In other embodiments, the sheath fluid is dispersed or removed from both the flow system and the submersible flow imager and is released into the surrounding environment. This embodiment may be of particular use in cases where the sheath fluid comprises seawater or any other non-toxic fluid or liquid (e.g., salt solution, fresh water). The removal of the particles after analysis allows for the efficient use of anti-fouling agents, which help the system operate for longer periods of time without the need for maintenance or cleaning. In one embodiment, the use of anti-fouling agents results in the submersible flow imager being capable of being used for at least 6 months without maintenance. In another embodiment, the use of anti-fouling agents results in the submersible flow imager being capable of being used for at least 8 months without maintenance. In still another embodiment, the use of anti-fouling agents results in the submersible flow imager being capable of being used for at least 12 months or more without maintenance.

In one embodiment, the flow system's operation comprises the sheath fluid and the sample to be analyzed (which in one embodiment is seawater comprising particles) are forced through a pair of 0.2 μm filter cartridges (e.g., Supor; Pall Corp.) by a gear pump (Micropump, Inc. Model 188 with PEEK gears) and flow through a conical chamber to a quartz flow cell.

The Sample Intake Means.

The sample intake means imbibes a sample from the aquatic environment and delivers it, via the pumping means, to the delivery means. The aquatic environment may comprise any environment around the submersible flow imager that comprises a liquid, including but not to limited fresh water environments, ponds, lakes, streams, oceans, rivers, and marine environments. Any suitable means known in the art may be employed as a sample intake means. In one embodiment, the sample intake means subsystem comprises an open tube and inline filter. The sample intake means subsystem should be designed such that it prevents imbibition of particles larger than the flow cell channel and does not damage fragile cells or colonies that the sample intake means subsystem imbibes. In some embodiments, the sample intake means comprises a filter to prevent imbibition of unsuitably large particles. In further embodiments, the filter is coated or fabricated with an antifouling surface. Antifouling surfaces may be surfaces comprising copper or an antifouling paint.

The sample intake means subsystem should not detrimentally introduce air bubbles or produce flow shear likely to damage fragile cells or colonies.

The Pumping Means.

The pumping means subsystem provides the motive force for imbibition of a liquid from the environment and transfer of the imbibed sample through the flow system. Any suitable means known in the art may be employed as a pumping means (e.g., a peristaltic pump, positive displacement pump, negative displacement pump, metering pump, etc.) and may function through the use of positive or negative pressure. In one embodiment, the pumping means comprises a syringe pump.

The Particle Suspension Means.

In some embodiments, the flow rate of the sample through the flow path is low (e.g., approximately 0.1 to 1 ml/min, 1 to 2 ml/min, 2 to 3 ml/min, up to 5 ml/min, or up to 10 ml/min). Therefore, one feature of the submersible flow imager is that the imbibed particulates pass through the system without settling out before reaching the detector. In embodiments that comprise fully vertical configurations, particle settling is not generally a concern since the flow is downward through a straight flow path. In such case, the particle suspension means subsystem is considered to be the liquid of the imbibed sample itself. However, in embodiments that any aspect of the flow system being located in a non-vertical orientation, particle settling may be a significant issue. Embodiments where any portion of the flow system is located in non-vertical orientation include, but are not limited to, low relief embodiments where the detection path may be in a different orientation than the delivery means. In embodiments where any one or more element of the flow system is in a non-vertical orientation, to ensure the particle suspension remains substantially intact, the continued suspension of the particles is ensured through exposure of the sample to turbulent flow or by moving particles at a velocity high enough to ensure that the particles do not settle while in the flow path. In one embodiment, the use of a syringe pump may itself be adequate to impart turbulence and/or the necessary velocity, when one or more element of the flow system is in a non-vertical orientation, to prevent particles from settling. In an alternative embodiment, a mixing device, including but not limited to a magnetic stirring bar, may be placed within the flow path at points of low velocity. In an embodiment, the magnetic stirring bar is located in the barrel of the syringe. In embodiments with a magnetic stirring bar, the magnetic stirring bar may be moved by an external magnet connected to a motor or solenoid. The presence of a mixing device, such as a magnetic stirring bar, within the syringe may preclude complete injection of the sample volume, but this is unimportant if the sample is well mixed. In yet another embodiment, the particle suspension means comprises a syringe pump and a magnetic stirring bar. In yet another embodiment, the particle suspension means comprises rotation of the syringe about its axis of movement. In other embodiments, particle suspension means comprises the tubing/piping/etc., which comprises the flow path, having a cork-screw shape. In a further embodiment, the wave action of the body of water in which the submersible flow imager is located comprises the particle suspension means. The particle suspension means can comprise any device or method that is known in the art for maintaining particles in suspension, whether through imparting turbulence into the sheath fluid containing the particles in suspension or otherwise.

In still another embodiment, the particle suspension means comprises the addition of a liquid to mix with the sample, to assure particle suspension. The liquid to be mixed with the sample can be any liquid that causes the suspended particles to be closer to neutral density (to float) than in its absence. Non-limiting examples of suitable liquids to be mixed with the sample include high-viscosity, hydrophilic liquids, such as: glycerol, isotonic sugars, polyethylene glycols, and other neutral surfactants. In some embodiments, the particle suspension means is a viscous suspending medium. In other embodiments, the viscous suspending medium that comprises the particle suspension means can also be used to carry the staining medium.

The Delivery Means.

The delivery means subsystem comprises the portion of the flow path connecting the pumping and/or the particle suspension means to the detection path and the delivery means is capable to transferring the liquid suspension from the sample intake means to a detection system. The delivery means receives the sample from the pumping means and then delivers the sample to the detection path. In some embodiments of the submersible flow imager where all aspects of the submersible flow imager are substantially vertical, the exit port of the pumping means comprises the delivery means. In other embodiments, where at least one element of the flow system is in a non-vertical orientation, portions of the flow path are non-vertical, and may occur at any suitable orientation relative to the detection path, which is substantially vertical. In these embodiments, the tubing/piping/etc. which comprises the flow path will have a directional change prior to connecting to the detection path. In some embodiments, the delivery means are designed so that, in combination with the particle suspension means, the particles to be analyzed do not settle out of the flow path.

The Detection System.

The detection system functions to detect one or more aspects of the suspended particles, which comprise the sample, passing through said detection system. The detection subsystem comprises the detection path, which comprises the portion of the flow path which carries the suspended particles into the detection interface subsystem. The detection interface subsystem comprises the core. The detection system also comprises the optical system and the sorting system. In one embodiment, the detection path and detection interface are substantially oriented vertically (e.g., in the direction of gravity), with a flow path contiguous with the delivery means and comprises the detection system. In other embodiments, the detection path and the detection interface are positioned in a non-vertical orientation. In yet another embodiment, the detection path and the detection interface are substantially horizontal.

The detection interface subsystem comprises the core, the sheath, and a flow cell. In one embodiment, the core is hydrodynamically focused. In one embodiment, the flow cell is a light transmitting flow cell. The light transmitting flow cell may be made of quartz, glass, fused silica, or plastic, depending on the means of detection and the aspects of the sample particles that are being detected. In another embodiment, the flow cell is opaque (e.g., dark or adapted to exclude at least some light).

In operation, the sample particle suspension is injected into the detection path by injecting, via the delivery means, the sample particle suspension into a flowing particulate-free sheath fluid. In some embodiments, in order to ensure the suspended particles to be sampled remain concentrated within the center of the sheath flow and pass through the flow cell in a correct orientation, the detection path and detection interface subsystem are maintained substantially vertically and fluids move through the detection path from top to bottom (with the flow of gravity). In one embodiment, the sheath fluid passes through a sheath filter to remove particulates or debris from the sheath fluid prior to entrance to the flow cell. In other embodiments, the detection path and the detection interface subsystem are positioned in a non-vertical orientation. In a further embodiment, the detection path and the detection interface subsystem are positioned so that they are in a substantially horizontal orientation. While moving within the detection path, the particles are exposed to the detection means. In some embodiments, the detection means includes exposure to an excitation wave length of light to stimulate the emission of light in the presence of an appropriate detector. In other embodiments, alternative detection means and systems besides fluorescence may be employed. Alternative detection means and systems that may be used include, but are not limited to, light scattering detection, conductivity detection, and any other detection system which is known in the art. The detection means include, but are not limited to, lasers, LEDs, or other focused light sources.

Sheath flow within the flow path is established through the use of the sheath fluid, which may be continuously recycled and reused. The term recycled, as it pertains to sheath fluid, means any re-use of sheath flow, and includes, but is not limited to, filtered sheath fluid or non-filtered sheath fluid. In some embodiments, the sheath fluid is recycled and reused a determined number of cycles through the submersible flow imager. In one embodiment, the sheath fluid is recycled and reused for 1 cycle. In another embodiment, the sheath fluid is recycled and reused for up to 10 cycles. In still another embodiment, the sheath fluid is recycled and reused for up to 20 cycles. In yet another embodiment, the sheath fluid is recycled and reused for up to 30 cycles. In another embodiment, the sheath fluid is recycled and reused for at least 30 or more cycles through the submersible flow imager. In an alternate embodiment, the sheath fluid is discarded after exiting the flow path (and therefore not recycled and reused), depending on the specific needs of the application as described elsewhere herein. In one embodiment, the sheath fluid is continuously recycled and reused. In another embodiment, the sheath fluid is discarded after exiting the detection path. In another embodiment, the sheath fluid is discarded after exiting the sorting system. In yet another embodiment, the sheath fluid is continuously recycled except when the sheath fluid contains a stain, which is added by the detection system. The flow cell comprises a channel. The channel comprises a core, and the core is surrounded by the sheath fluid. The channel flows into the flow cell. In one embodiment, the flow cell channel is modified from that of the Becton Dickinson (BD) FACSCalibur™ flow cell to produce a wider ribbon-shaped core, thus increasing the sample volume that can be processed without increasing the core thickness.

The submersible flow imager comprises at least two valves. The valves can be, but are not limited to, solenoid valves, electromechanically-operated valves, and 2-way valves. The first valve is located between the exit from the flow cell and the entrance to the sheath filter. The second valve is located at a penetration point through the underwater housing of the submersible flow imager. The purpose of the second valve is to provide a source of seawater for new, as opposed to recirculated, sheath fluid (see Example 2—Detection of Stained Phytoplankton).

The detection system detects one or more aspects or features of the suspended particles passing through the detection interface subsystem of the flow system. In some embodiments, the sample particles comprise a fluorescent aspect, either inherently or after being exposed to a dye or stain capable of causing part or all of the particle to fluoresce. Any detectable feature of the particles may be detected through use of an appropriate detection means, which are known in the art. In one embodiment, the detection system comprises a device to detect fluorescence. The detection system interfaces with the detection path subsystem and the detection interface subsystem. In one embodiment, the flow cell comprises a light transmitting flow cell.

The detection system further comprises a combination of video and flow cytometric technology, which comprises the optical system, to both capture images of organisms for identification and measure chlorophyll fluorescence associated with each image. In one embodiment, images can be automatically classified with software based on a support vector machine, while the measurements of chlorophyll fluorescence allow the efficient analysis of phytoplankton cells by triggering on chlorophyll-containing particles. In an embodiment, quantitation of chlorophyll fluorescence in large phytoplankton cells enables the discrimination of heterotrophic and phototrophic cells.

Electronics System.

The detection system and flow systems are electronically controlled by an electronics system that comprises: at least one controller, a data acquisition device, a power source, and a data processor. In one embodiment, the electronics system also comprises telemetry devices. In one embodiment, the electronics system also controls the anti-fouling means.

In another embodiment, the electrical system comprises programmable operations. In another embodiment, the programmable operations comprise at least one of the following: data acquisition, transfer of data (e.g., data transmission capacity) (to shore or to any other device capable of receiving data, whether located on shore or not, such as a mooring, buoy, or vessel), adjustment of sampling frequency, adjustment of rate of sample injection, injection of internal standard beads, flushing the flow cell with an anti-fouling or cleaning agent, flushing the sample tubing with the cleaning agent, flushing the sheath with cleaning agent, backflushing the sample tubing to remove potential clogs, flushing the flow cell to remove air bubbles, adding anti-fouling agents to the sheath reservoir to prevent bio-fouling of the internal surfaces of the submersible flow imager, focusing the optical system, or focusing of the imaging objective lens (or lenses).

Anti-Fouling Means.

All instruments exposed to the aquatic environments are subject to bio-fouling. This problem can be acute for optical sensors since biofilms and macro-organisms can interfere with light transmission and can ultimately render sensing modalities ineffective. Bio-fouling can also obstruct or contaminate sample particle intake. In one embodiment, the submersible flow imager prevents bio-fouling of intake and internal surfaces through a multi-pronged approach. First, the flow cell is protected from contact with seawater by containing the sample within the sheath fluid. Further, the sheath fluid is treated so that it remains particle-free. In one embodiment, the sheath fluid is treated by recirculation of the sheath fluid through particle filters. In another embodiment, the sheath fluid is treated by regular injection of a biocide or other suitable chemical. In one embodiment, the sheath fluid is treated by regular injection of chlorine. In yet another embodiment, the sheath fluid is treated by recirculation of the sheath fluid through particle filters and regular injection of a biocide. In another embodiment, the sample particle intake and the internal tubing of the submersible flow imager are subject to automated periodic cleaning cycles, accomplished by back-flushing with appropriate cleaning agent. Typical cleaning agents include acids, bases, or surfactants. In one embodiment, the cleaning agent comprises sodium hypochlorite. In other embodiments, a coating is applied to one or more internal surfaces of the flow system, such as the flow cell, to prevent the attachment of biofilms. In one embodiment, the coating comprises an anti-fouling coating. In another embodiment, the coating comprises a non-stick coating. Non-limiting examples of non-stick coatings include: low friction coatings, hydrophobic coatings, and zwitterionic coatings. In another embodiment, the flow system comprises a UV-emitting device, and the UV-emitting device functions to reduce bio-fouling by appling ultraviolet in the direction of the flow cell, delivery means, the detection system, the pumping means, or any other suitable surface within the flow system. In one embodiment, the ultraviolet light is between approximately 200 nm and approximately 295 nm. In another embodiment, the ultraviolet light is between approximately 265 nm and approximately 295 nm. The UV-emitting device may be programmed to operate for a period of time at a specific duty cycle and then to de-activate during other specific duty cycles. In one embodiment, the UV-emitting device is de-activated during the duty cycle for the acquisition and measurement of samples in the flow cell.

Use of the Submersible Flow Imager.

Generally, a sample (e.g., seawater) comprising a liquid suspension of particles is injected into the center of a sheath flow of particle-free water; all the particles are thus substantially confined into a core flowing through the center of the flow cell, which ensures that each particle is in focus as it passes through the detection system, which comprises the optical system. In an embodiment, the sheath fluid is recycled through a filter cartridge, which removes the sample particles after they have been analyzed. Recycling the sheath fluid and removing the sample particles after analysis allows for the efficient use of anti-fouling agents so the system can operate for months or more at a time without the need for maintenance or cleaning.

In one embodiment, the submersible flow imager is contained in a watertight housing. In another embodiment, the housing is capable of remaining watertight when submerged to water depths of at least approximately 50 m without deformation of the submersible flow imager or its components. In another embodiment, the housing is capable of remaining watertight when submerged to water depths of at least approximately 100 m without deformation of the submersible flow imager or its components. In another embodiment, the housing is capable of remaining watertight when submerged to water depths of between approximately 100 m and approximately 1,000 m without deformation of the submersible flow imager or its components. In still another embodiment, the housing is capable of remaining watertight when submerged to water depths of between approximately 1,000 m and 5,000 m without deformation of the submersible flow imager or its components. In yet another embodiment, the housing is capable of remaining watertight when submerged to water depths of at least approximately 6,000 m without deformation of the submersible flow imager or its components. In another embodiment, the housing is capable of remaining watertight when submerged to water depths of up to approximately 11,000 m or more without deformation of the submersible flow imager or its components. In one embodiment, the submersible flow imager is operated continuously and autonomously, under the direction of a computer whose programming can be modified by a remote operator. In such embodiments, programmable operations include but are not limited to: data acquisition, data transfer to shore or to a device capable of receiving such data transmission regardless of whether the device is located on shore, activating and/or controlling motors, activating and/or controlling valves, adjustment of sampling frequency, adjusting rate of injection of samples, injection of internal standard beads, flushing the flow cell and/or sample tubing with detergent, backflushing the sample tubing to remove potential clogs, adding biocide to the sheath reservoir to prevent bio-fouling of the internal surfaces, adjusting the UV-emitting device duty cycle, focusing the imaging objective lens, providing dye and/or other additives, recycling of sheath fluids, and/or similar control activities.

Flow Rate. In one embodiment, flow rates of sample particles through the submersible flow imager range from approximately 0.1 ml/min to approximately 1 ml/min, and in some cases up to approximately 5 ml/min or more.

Bubble Minimization Assembly and/or Suppression System.

Under most circumstances, the presence of bubbles within the flow system is undesirable because it disrupts the flow pattern within the flow cell, causing some cells to fail to trigger the detection system or be out of focus. In one embodiment, the submersible flow imager comprises a system and/or assembly to minimize the introduction of bubbles or the generation of bubbles within the imbibed samples and to remove bubbles from the flow cell. In one embodiment, the bubble minimization assembly comprises regulation of the sample aspiration rate. In another embodiment, the bubble minimization assembly comprises capabilities for aspiration from above the flow cell and the expulsion of the resulting fluid to the outside of the instrument. In another embodiment, a modified vacuum aspirator is used to generate a vacuum while the vehicle comprising the submersible flow imager is in motion. A vacuum so generated is used to draw dissolved air from the imbibed sample in order to reduce the possibility of bubble formation. In some embodiments, the bubble minimization assembly comprises a bubble removal port which further comprises one or more tubes and a valve for the syphoning and/or expulsion of the bubble-containing fluid to the outside environment. In one embodiment, the valve in the bubble minimization assembly comprises a T-valve. In one embodiment, the flow system software is capable of detecting the presence of bubbles and performing automatic and/or scheduled expulsion of any bubble-containing fluid in the flow system. In a further embodiment, the software is capable of detecting the presence of bubbles prior to the introduction of bubbles into the flow cell to minimize corruption of data.

Automated Staining System.

Use of Stains.

In one embodiment, the detection system of the submersible flow imager comprises equipment for the automated introduction of liquid or dissolved stains for the purpose of treating sample particles to enhance the analysis capabilities of the detection system following the detection of the degree of staining of the sample particles by the detection system. The stain utilized should be a stain that will remain stable within the stain reservoir until used. Additionally, the stain used should be a stain that is used in sufficiently small amounts to ensure the capability of the submersible flow imager to stain many samples on long deployments. In some embodiments, the volume of stain used per sample is approximately 10 to 20 μL and the stain reservoir contains approximately 100 to 200 mL, so that the number of samples that can be stained is approximately 5,000 to 20,000 (or approximately 69 to 278 days of continuous analysis at a rate of approximately 0.25 mL of seawater/min.). Other embodiments may employ larger stain reservoirs up to approximately 500 mL and in some instances up to approximately 1 L or more.

Liquid or dissolved stain is introduced from a stain reservoir into the mixing chamber section of the delivery means flow path through the use of a solenoid activated micropump.

In one embodiment, when a stain is used, the sheath fluid will be discarded after a single passage through the detection path. However, in other embodiments, the optional control of sheath fluid recycling is accomplished through the use of two solenoid activated valves (see Example 2—Detection of Stained Phytoplankton) as disclosed above. In some embodiments, the submersible flow imager further comprises a variable filter in the detector portion of the detection system to change the wavelengths of light being detected. The variable filter may comprise one or more filters of variable wavelengths capable of being placed into the detection light path through the use of a motor or solenoid.

Fluorescent Staining Systems.

In another embodiment, the detection system comprises a fluorescent staining system. Many fluorescent staining systems, which are known in the art, may be used depending upon the specific needs of the application. Stains may be selected and used by employing principles similar to those of the LIVE/DEAD® products provided by Life Technologies (3175 Staley Road, Grand Island, N.Y. 14072). Non-limiting examples of potentially useful Life Technology stains include:

(i) membrane-permeant calcein AM, which is cleaved by esterases in live cells to yield cytoplasmic green fluorescence,

(ii) membrane-impermeant ethidium homodimer-1, which can be used to label nucleic acids of membrane-compromised cells with red fluorescence,

(iii) DiOC18(3), which is a green-fluorescent membrane stain that is used to stain target cells prior to exposing the target cells to propidium iodide,

(iv) SYBR® 14 nucleic acid stain, which labels live cells with green fluorescence,

(v) membrane-impermeant propidium iodide, which labels the nucleic acids of membrane-compromised cells with red fluorescence,

(vi) C12-resazurin, which is reduced to red-fluorescent C12-resorufin in metabolically active cells,

(vii) SYTOX® Green dye, which is a cell-impermeant green-fluorescent nucleic acid stain, and which can be used to stain cells with compromised plasma membranes (usually late apoptotic and necrotic cells); in this assay, dead cells emit mostly green fluorescence and healthy, metabolically active cells emit mostly red fluorescence; injured cells exhibit reduced red and green fluorescence,

(viii) Hoechst nucleic acid stain,

(ix) Fluorescein diacetate (FDA), which stains cells with intact membranes and esterase activity, and

(x) LysoTracker Green®, which stains cells with acidic vacuoles.

Low Relief Configuration

Design.

In another embodiment, the submersible flow imager is fabricated according to a modularized design, employing the inventive detection path in a substantially vertical orientation with the remainder of the device positioned substantially lateral to the substantially vertical detection path and lower than the top of the detection path; in other embodiments, all aspects of the submersible flow imager, including the detection path, are in a substantially horizontal orientation. Both embodiments are referred to as low relief configuration embodiments. In embodiments where the detection path is substantially vertical, the overall height of the submersible flow imager is primarily dictated by the height of the detection path and any clearance required for the attachment of the delivery path and the device housing. Such configurations can result in a maximum device height of approximately 48 cm or less. In some embodiments, the maximum device height is less than approximately 36 cm, 25 cm, 20 cm, 19 cm, 17 cm, 15 cm, or 10 cm. In alternative embodiments, the laterally positioned systems may extend above and/or beyond the upper reaches of the detector flow path, but below the height required if the entire flow path was disposed in-line and substantially vertical.

In a specific embodiment, the submersible flow imager is configured for low relief (also referred to herein as “IFCB-LR”) for operation on platforms and vehicles with instrument height restrictions. One example of such a platform/vehicle is the Wave Glider 3, whose payload bay's dimensions are approximately 59 cm×38 cm×19 cm high. Accordingly, in one embodiment, the submersible flow imager height is no more than approximately 17 cm.

In one embodiment, the submersible flow imager's flow cell should remain substantially vertically oriented (with flow downward) so that the sinking of particles as they flow through the detection system does not affect their trajectory (and hence signal detection and image focus). The flow cell assembly in the submersible flow imager is approximately 17 cm tall, so use of the flow assembly is compatible with installation of the submersible flow imager in a Wave Glider 3 payload bay. In another embodiment, the submersible flow imager's flow cell should remain substantially horizontally oriented, and the sinking of particles is prevented through the use of the particle suspension means.

In one embodiment, the design of the submersible flow imager comprises the sample syringe pump being located substantially vertically above the flow cell. In some embodiments, the syringe pump is approximately 30 cm in length, therefore locating the sample syringe pump substantially vertically above the flow cell is not well suited for most small vehicles and platforms. When the submersible flow imager is to be deployed in a small vehicle or platform, the syringe pump may be positioned substantially horizontal to the flow cell or replaced by a different method of injecting sample water.

In one embodiment, the optical system of the detection system comprises a trigger laser, an illumination system, and a detector system. The illumination system comprises a flash lamp and a condenser. In a further embodiment, the illumination system comprises a Xenon flash lamp. The detector system comprises an objective lens, photomultiplier, and a camera. The optical components of the optical system occupy a space of approximately 40×16×9 cm, which will fit in the Wave Glider 3 payload bay. In order to meet this space restriction, the image as shown in FIG. 1 must be re-oriented from vertical to horizontal. In another embodiment, the electronics module, which occupies a space of approximately 13×13×12 cm, and the fluidics module, which occupies a space of approximately 16×12×12 cm, are relocated to meet the space limitations of the Wave Glider 3 payload bay. In an alternate embodiment, in situations where payload space is available in fixed size bays, some of the modules can be located in separate bays other than the bay in which the optical components are located. In yet another embodiment, if the vehicle or platform has integrated processors and/or data storage capacity, the electronics module can be further subdivided to offload these functions from the instrument to the platform.

Automated Sorting System.

Image-Based Cell Sorting and Preservation.

The detection system further comprises a sorting system. Flow cytometric cell sorting, using fluorescence and light scattering to classify cells to broad groups, has become a powerful tool for microbial ecologists, especially in the context of advances in genomic methods. The ability to sort with genus or species resolution would be even more powerful; for example, species-sorted samples from different stages of a bloom would provide unprecedented opportunities for transcriptomic studies to provide a direct link between species and gene expression as a function of environmental condition. To date, these methods have not generally been available for analysis while at sea. Additional embodiments of the sorting system are described below in Example 4). In one embodiment, the sorting system is integrated into a low relief configuration for operation in the water. In another embodiment, the sorting system is either attached to the housing or disposed within the hull of the vehicle in which the IFCB is mounted. In yet another embodiment, the sorting system is located outside of the housing of the submersible flow imager.

A prototype imaged-based cell sorter function has been incorporated into a submersible flow imager. The major challenge in this approach is that the speed of image-based sorting is fundamentally limited by that of image processing and classification computations. Currently, image processing cannot be completed in the fraction of a second available between a sample particle's passage through the detection region and the sorting location. Accordingly, in one embodiment, the submersible flow imager comprises a two-step sorting procedure. The first step comprises the use of a commercially available BD FACSCalibur sort flow cell assembly, in which a ‘catcher tube’ is positioned below the flow cell. At rest, the catcher tube opening is located outside the core, with the particle-free sheath flowing through it. When a sample particle with the desired fluorescence and/or light scattering signature is detected, a piezo element pushes the catcher tube out into the core, so that the catcher tube will be able to collect the particle at the correct moment after the particle leaves the flow cell and the sample particle of interest goes into the tube. The second step comprises classifying the captured image. In this embodiment, the imaging sorter comprises a miniature solenoid valve. In the imaging sorter, a miniature solenoid valve, which is located below the catcher tube, shuts off the flow after the cell is captured and the sample particle then waits in the catcher tube while the image is classified. If the image does not match pre-determined target criteria, the sample particle is sent to the waste stream and the sorting process starts over. If the image classification does satisfy the target criteria, a separate solenoid directs the flow to a capillary above a well plate. A second laser and detector assembly is used to verify that a sample particle was actually captured and to enable the correctly timed release of a drop the sheath fluid containing the sample particle to be preserved into the well.

In one embodiment, fluorescence measurements and image capture takes place in a BD FACSCalibur™ sort flow cell. In another embodiment, the catcher tube is controlled by FACSCalibur™ circuitry (not shown). In yet another embodiment, the solenoids, capillary fluorescence detector, waste catcher (for removing the sheath flow in between sorted cells), and the well plate X-Y translator are controlled by a PIC microprocessor (not shown).

The 2-step image-based sorting system described above is functional, but slow, because the sorting decision depends on computationally intensive image processing and classification algorithms which can require up to several seconds, depending on the image size. During the time the sorting decision is occurring, no other sample particles can be processed. Additionally, in the 2-step process described above, the sorted drops are exposed to the air and only a single plate of samples can be sorted, neither of which is optimal for operation in situ. Finally, in the 2-step imaging-based sorting process described above, a relatively large volume of seawater (approximately 1 ml) accompanies each sorted particle, making it difficult to manually check sorting efficiency. Further, the relatively large volume of seawater that accompanies each sorted particle can cause interference problems in molecular assays. Accordingly, in another embodiment, the second stage of the sorting process uses an alternate strategy. In this embodiment, the second stage of the sorting process is based on methods that allow for parallel rather than serial processing of sample particles.

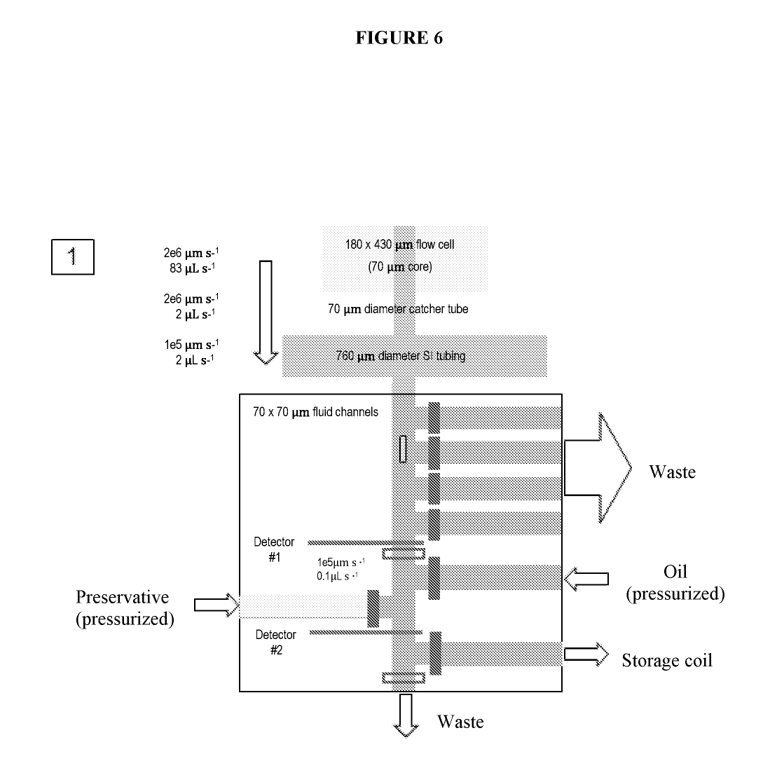

In this alternate embodiment, where the second stage of the sorting process enables parallel processing, the second stage of the image-based sorter is replaced by a sorted drop storage module (see FIG. 6), which allows the fluorescence-based and image-based sorting steps to be separated in time. In this embodiment, every potential target, e.g., those sample particles with high-fluorescence, scattering, and/or long signal duration, etc., is captured by the real-time sorting step, which was step one, and stored, so that more specific image-based sorting can be carried out in a separate procedure later (after image analysis, classification, and identification of target groups). In one embodiment, the storage of sorted particle samples, that are retained from the first step (as well as the subsequent image-based sorting), is carried out using water-in-oil technology in which stable fluid compartments are formed and manipulated, each fluid compartment containing a sorted sample particle. Each sorted sample particle from the initial sorting step is injected, along with a small parcel of water, into an oil stream in a microfluidic channel, to form a stable chain of water segments, each water segment being separated from other water segments by oil, whose contents are knowable from the corresponding saved images. The water segments can be stored in a compact coil of tubing for an extended period of time, during which time the image processing and classification can be completed. In another embodiment, the storage of sorted particle samples, that are retained from the first step (as well as the subsequent image-based sorting), is carried out using a reel of tape with micro-wells, where each well is sealed containing a fluid drop and associated sorted sample particle. These steps are carried out in the instrument deployed in the field. Later, back in the shore laboratory, the desired water segments are retrieved from the coil of tubing or reel of micro-well tape, allowing specific target groups to be isolated, as desired. In this embodiment, the image-based sorter prototype utilizes a second stage capillary detection system, whose function is to verify that the preliminary sort captured a sample particle and then time that sample particle's release into a well. This capillary detection system is the “front end” of the emulsion system of cell sorting. In one embodiment, the microfluidics system processes drops more rapidly than the camera's maximum frame rate, which is 30 Hz, so the emulsion based sorting system is capable of sorting sample particles based on their images at rates that are essentially the same as those of analytical sampling.

In one embodiment, the oil and water segments are stored in coiled tubing. In another embodiment, for transcriptomic analysis, the sample stream is mixed with the RNA preservative (e.g., ethanol). The RNA preservative can be any substance that is now known or in the future determined to preserve RNA. Later before the injection of the sample along with the small parcel of water into the oil stream to preserve RNA. In this embodiment, the storage chamber temperature can be controlled, if necessary.

The submersible flow imager's sampling rate is limited as a consequence of the stringent requirements of the imaging that enables the resolution of cells to the genus or species level because the depth of the focus of the microscope objective is only approximately a few microns. In one embodiment, the sample stream is hydrodynamically focused to form a ribbon-shaped core, but to achieve a core this thin the sample flow rate is limited to approximately 0.25 mL/min. This sampling rate is 5 times faster than that of the FCB, which measures Synechococcus integrated optical properties, but the difference in cell concentration between Synechococcus and large diatoms is typically far more than 5-fold, even when the diatom biomass far outweighs that of the picoplankton; Synechococcus typically occurs at approximately 10⁴-10⁵ cells/mL, while all the diatom species combined often comprise only approximately 10³ cell s/mL.

Similar sampling limitations apply to many methods in plankton ecology. In the case of the submersible flow imager, simply pumping samples faster degrades the quality of the images because the thickness of the sample core in the flow cell is determined by the relative rates of sample flow and sheath flow; therefore, if the core gets thicker, some cells will no longer be at the proper distance from the microscope objective. However, increasing the sheath flow to correct the core thickness causes the velocity to increase, which results in blurred images.

An alternative to increasing the instrument's sampling rate is to pre-concentrate samples by removing most of the water from the sample, leaving the sample particles behind. Pre-concentration by settling, centrifugation, or filtration is a routine part of land-based microscopic or bulk constituent analysis. Settling and centrifugation are not optimal for in situ operations. Filtration is only semi-quantitative when the samples need to be re-suspended for analysis, as sample particles or cells can be destroyed and/or stick to the filter. In one embodiment, the submersible flow imager detection system further comprises filtration for pre-concentration. Alternatively, acoustic standing waves can be a solution for in-line concentration of flowing samples. Accordingly, in an alternate embodiment, the delivery system comprises acoustic focusing devices that create acoustic standing waves for in-line concentration of flowing samples. In yet another embodiment, the acoustic focusing device comprises a piezo electric drive.

Though acoustic focusing devices can take several forms, among the simplest is a piezo electric drive that generates a standing wave in a rigid cylindrical tube. As the standing wave in this case is generated by the tube, it has a complex structure that leads to a single pressure node that radially focuses particles to the center of capillary flow. This approach has been applied to land-based flow cytometry, where tests with plastic beads and mammalian cells demonstrated that an acoustic system can achieve particle focusing comparable to that of a conventional hydrodynamic system, and the system has been incorporated into a commercial flow cytometer (Attune, Applied Biosystems).

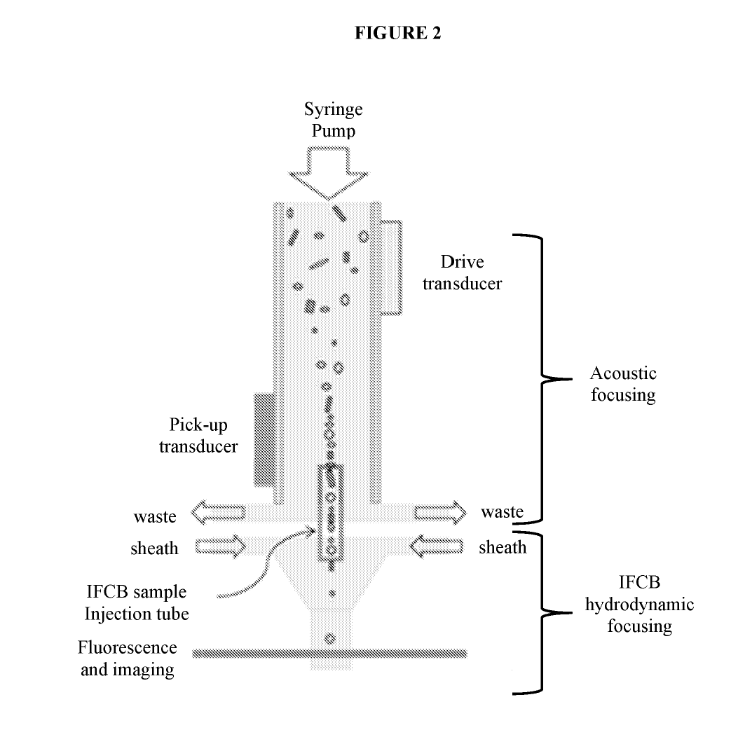

In one embodiment, the submersible flow imager further comprises an acoustic focusing element, located just prior to the flow cell, to increase its sampling rate (FIG. 2). In this embodiment, the submersible flow imager's hydrodynamic focusing system is retained because it prevents bio-fouling and because it maximizes the in-focus sample volume by creating a ribbon-shaped core flow. The sampling syringe pump pushes seawater at approximately 4 mL/min through an approximately 2 mm internal diameter (also referred to as “ID”) tube, which is approximately 190 mm long and equipped with an acoustic transducer. At the outflow end of the tube, all the particles are in approximately the center 40 μm of the flow; this part of the flow enters the submersible flow imager's sample injection needle (internal diameter approximately 380 μm), with the remainder of the flow sent overboard. Since the submersible flow imager's current sample rate is approximately 0.25 mL/min, the modification represented in this embodiment is an approximate 16-fold increase in sample rate. In other embodiments, even higher concentrations of sample particles can be achieved by increasing the cross-sectional diameters of the acoustic focusing channel. In some embodiments, the submersible flow imager employs an acoustic focusing element or system (such as the device described in U.S. Pat. No. 8,830,451, which is incorporated in its entirety by this reference) in a suitable manner as known by one skilled in the art.

For scientific purposes where the current data density is already sufficient, increasing the sampling rate is still advantageous because it allows for a decreased duty cycle, which is important for power conservation during moored deployments. If power is not a consideration, the faster instrument is able to complement standard sampling with alternative functions, such as cell sorting or specialized measurements with fluorescent probes. Increased sample particle concentration also allows for reduced velocity of the sheath flow, allowing longer-duration camera exposures, which in turn allows for replacement of the Xenon flash lamp in the illumination system by a less powerful LED light source, thereby reducing space, power, and expense. Accordingly in one embodiment, the illuminating system further comprises a LED light source. In yet another embodiment, the illuminating system comprises a pulsed laser.

Applications.

Useful applications for the submersible flow imager include: (1) detection of fluorescent or fluorescent-stainable toxins or chemicals taken up by the sample particle; (2) detection of intracellular calcium levels within sample particles using calcium sensitive dyes; (3) assessment of the metabolic state of specific sample particles; (4) detection of NAD/NADH content of sample particles; (5) species identification; (6) the presence of chlorophyll; (7) analysis of cell-to-cell variability; (8) comparative analysis between morphology and phenotype in organisms; and/or (9) size-based fractionation of cells, particularly for studies of the life cycle of organisms.

### EXAMPLES

### Example 1

### Fully Vertical Flow Analyzer

In one embodiment, the flow system of the submersible flow imager is based on that of a conventional flow cytometer and comprises hydrodynamic focusing of a seawater sample stream in a particle-free sheath flow that carries cells in a single-file manner through a laser beam and then through to the camera's field of view.

In this embodiment, sheath fluid, plus the suspended sample, flows through a conical chamber to a quartz flow cell. The flow cell housing and sample injection tube is from a Becton Dickinson FACScan flow cytometer, but the flow cell is replaced by a custom cell with a wider channel. The channel dimensions of the custom flow cell are approximately 800×180 μm (Hellma Cells, Inc.). Since the FACScan objective lens housing, which normally supports the plastic flow cell assembly, is not used, an aluminum plate that is approximately 3.175 mm thick is bolted to the assembly. The aluminum plate is used to support the flow cell assembly. The sheath fluid is recycled by using a gear pump (Micropump, Inc. Model 188 with PEEK gears) to force the sheath fluid through a pair of approximately 0.2 μm filter cartridges (Supor; Pall Corp.), one of which is positioned before the flow cell and the other after the flow cell.

The sample is imbibed through an approximately 130 μm Nitex screen, which prevents flow cell clogging. The Nitex screen is protected against bio-fouling by an approximately 1 mm copper mesh. After the sample is imbibed through the Nitrex screen, the sample is injected through a stainless steel tube (approximately 1.651 mm outer diameter (OD), approximately 0.8382 mm internal diameter (internal diameter is abbreviated herein as “ID”); Small Parts, Inc.) into the center of the sheath flow in the cone above the flow cell by a programmable syringe pump (Versapump 6 with approximately 48,000 step resolution, using a 5 mL syringe with Special-K plunger; Kloehn, Inc.). The tubing is of PEEK material (approximately 3.175 mm internal diameter for sheath tubes, approximately 1.588 mm for others; Upchurch Scientific).

An 8-port ceramic distribution valve (Kloehn, Inc.) allows the syringe pump to carry out several functions in addition to seawater sampling. These include regular (approximately daily) addition of biocide to the sheath fluid to prevent bio-fouling, and regular (approximately daily) analyses of beads (approximately 20 μm or approximately 9 μm red-fluorescing beads, Duke Scientific, Inc.) as internal standards to monitor instrument performance. In addition, the sample tubing, which is not protected from bio-fouling by contact with biocide-containing sheath fluid, is treated with detergent (approximately 5% Contrad/1% Tegazyme mixture) during bead analyses (approximately 20 min/day) to remove fouling. Finally, the syringe pump is used to prevent accumulation of air bubbles in the flow cell, which may result from the degassing of seawater, since air bubbles could disrupt the laminar flow pattern. Before each sample is injected, sheath fluid is withdrawn from the sample injection needle and from the conical region above the flow cell, and discarded to waste. The biocide solution, suspended beads, and detergent mixtures are stored in separate 100 mL plastic bags with Luer fittings (Stedim Biosystems).

In one embodiment, flow cytometric measurements are derived from a red diode laser (SPMT, 635 nm, 12 mW, Power Technologies, Inc.) focused to a horizontally elongated elliptical beam spot by cylindrical lenses (horizontal=approximately 80 mm focal length, located approximately 100 mm from the flow cell; vertical=approximately 40 mm focal length, at approximately 40 mm). Each sample particle passing through the laser beam scatters the laser light, and chlorophyll-containing cells emit red (680 nm) fluorescence. One of these signals, usually chlorophyll fluorescence, is chosen to trigger a xenon flash lamp (Hamamatsu L4633) when the signal exceeds a preset threshold. The resulting approximately 1 μs flashes of light are used to provide Kohler illumination of the flow cell. The green component of the light, isolated by an approximately 530 nm bandpass filter, is focused into a randomized fiber optic bundle (approximately 50 μm fibers, approximately 6.35 mm diameter; Stocker-Yale, Inc.). At the fiber optic bundle exit, the light is collected by a lens, passed through a field iris, and focused onto a condenser iris, which is located approximately at the back focal plane of a 10×objective lens (Zeiss CP-Achromat, numerical aperture [N.A.] 0.25), which is in turn focused on the flow cell. A second 10× objective (Zeiss Epiplan, N.A. 0.2) collects the light from both the flash lamp illumination (green) and the laser (red, 635 nm scattered light and 680 nm chlorophyll fluorescence). The green and red wavelengths are separated by a dichroic mirror (630 nm short pass). The green light continues to a monochrome CCD camera (UniqVision UP-1800DS-CL, 1380×1034 pixels). The red light is reflected to a second dichroic mirror (635 LP), which direct the scattered laser light and fluorescence to separate photomultiplier (PMT) modules (Hamamatsu HC120-05 modified for current-to-voltage conversion with time constant=800 kHz; the PMT for laser scattering also incorporates DC restoration circuitry).

In an embodiment, the optical path is folded, by broadband dielectric mirrors (Thorlabs BB1-E02), on either side of the flow cell to conserve space. The flow cell assembly is fixed to the optical table, while the light source/condenser and objective/PMT/camera assemblies are each mounted on lockable translators (Newport Corp.), providing 3 degrees of freedom for adjustment. The objective focusing translator is remotely controllable. Optical mounting hardware is from Thorlabs, Inc.

In an embodiment, the imaging apparatus is controlled by a PC-104plus computer (Kontron MOPS-LCD7, 700 MHz), running Windows (Microsoft Corporation). Remote operation is carried out via Virtual Networking Computing software (www.realvnc.com) The camera is configured and the syringe pump is programmed by software provided by the manufacturers. All other functions (control, image visualization, and data acquisition) are carried out by custom software, written in Visual Basic 6 (Microsoft Corporation).

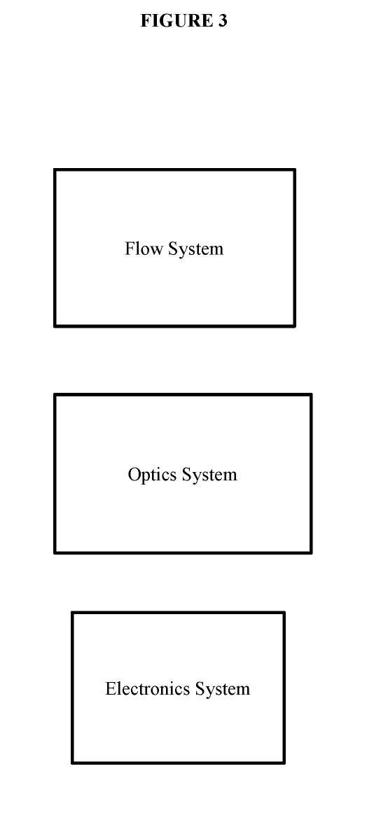

In one embodiment, a custom electronics board amplifies and integrates light scattering and fluorescence signals and also generates control pulses for timing purposes (FIG. 3). The signal from the triggering PMT, which is typically chlorophyll fluorescence, is split, with one part sent to a comparator circuit that produces a trigger pulse if the signal is larger than a preset threshold level. The other part of the signal, and the signal from the other PMT, are delayed by approximately 7 μs (by delay modules from a Coulter Electronics EPICS 750 flow cytometer) and then split and sent to paired linear amplifiers with approximately 25-fold different gains, which increase dynamic range, before integration (Burr-Brown AFC2101). The delay modules allow the pre-trigger portions of the signals to be included in the integration. The end of the integration window is also determined by the comparator, with the provision that the signal remains below the comparator threshold for approximately 20 μs; this allows signals from loosely-connected cells, such as chain diatoms, to be more accurately measured. Comparator output pulses are also integrated to provide an estimate of the duration of each signal. The PMT amplifier inputs are grounded by transistors during flash lamp operation, to avoid baseline distortion by the very large signals from the flashes (FIG. 3A, D).

In another embodiment, the trigger pulse is also sent to a frame grabber board (Matrox Meteor II CL) to begin image acquisition, and, after a delay of approximately 270 μs, to the flash lamp, which illuminates the flow cell for an approximately 1 μs exposure. Integration of light scattering and fluorescence signals is limited to approximately 270 μs to avoid contamination by light from the flash lamp, so integrated signals from particles, cells or chains of cells longer than approximately 600 μm are conservative estimates.

In one embodiment, a multifunction analog-digital (A-D) board (104-AIO16-16E, Acces I/O Products, Inc.) digitizes the integrated laser-derived signals and the duration of the triggering signals, produces analog signals to control the PMT high voltages, and carries out digital I/O tasks (e.g., motor control for focusing the objective and communication between software and hardware, i.e., inhibiting new trigger signals while the current image is being processed).

In an alternative embodiment, to minimize the resources needed for image data storage, the apparatus utilizes a “blob analysis” routine (Matrox Imaging Library) based on edge detection (changes in intensity across the frame) to identify regions of interest in each image. The subsampled images are transferred to a remote computer for storage and further analysis. For taxonomic classification, approach was developed based on a support vector machine framework and several different feature extraction techniques; this approach is described elsewhere (Sosik and Olson 2007), along with the results of automated classification of 1.5×10⁶ images obtained during the apparatus's test deployment in Woods Hole Harbor.

For each particle, 5-9 channels of flow cytometric signal data are stored (integrated and peak signals from 2-4 fluorescence and light scattering detectors, plus signal duration), along with a time stamp (approximately 10 ms resolution). Accumulated images and fluorescence/light scattering data are automatically transferred to the laboratory in Woods Hole approximately every 20 min. The data are analyzed using software written in MATLAB (The Mathworks, Inc.).

In practice, the apparatus can be deployed by divers, who bolt the neutrally buoyant approximately 70 kg instrument to a mounting frame located at approximately 4 m depth on the MVCO Air Sea Interaction Tower (http://www.whoi.edu/science/AOPE/dept/CBLAST/ASIT.html), and connect the power and communications cable, which is equipped with an underwater pluggable connector (Impulse Enterprise, Inc.).

Hydrodynamic focusing causes all the particles in a sample to pass through the submersible flow imager's detection system, allowing the calculation of particle concentration, to a first approximation, by dividing the number of triggers by the volume of water analyzed, as determined by the analysis time and the known rate of flow from the syringe pumps. However, this concentration is an underestimate, because during the time required to acquire and process each image, sample continues to flow through the flow cell, but no new triggers are allowed. The minimum time required by the camera for image acquisition is approximately 34 ms (i.e., approximately 30 frames/sec), but it was determined empirically that with image processing to locate and store the region of interest, at least approximately 86 ms was required by the system; very large particles required even more time. Therefore, the image processing period for each cell was measured using a software timer. By subtracting the sum of these periods from the total elapsed time, the amount of time that was actually spent “looking” for sample particles was determined and used to calculate the sample particle concentration in each syringe sample.

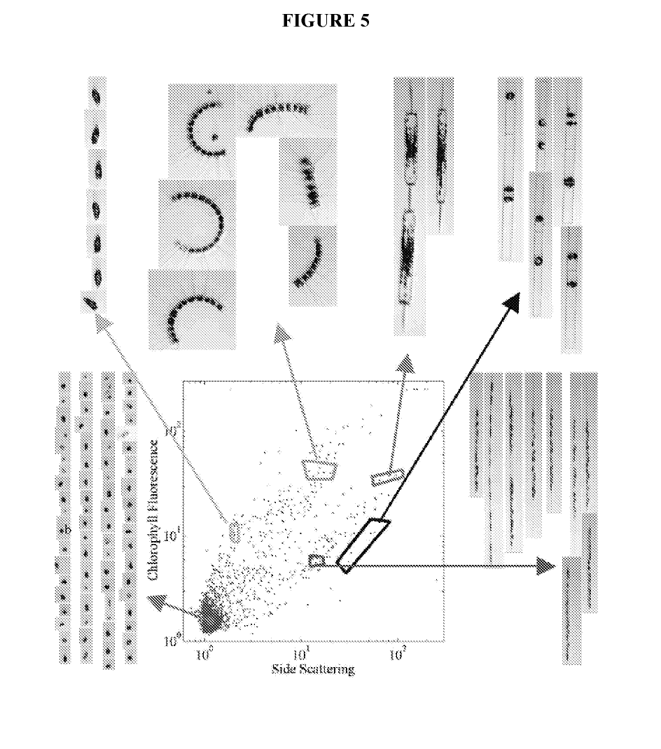

Analysis of seawater samples by the apparatus illustrates some advantages of the approach described herein over conventional flow cytometry and manual microscopic analyses. First, flow cytometric sorting of particles in seawater has shown that light scattering/fluorescence signatures are rarely sufficient to identify nano- or microplankton at the genus or species level. Discrete populations are rarely discernible in a plot of light scattering vs. fluorescence (e.g., see FIG. 5), and even if they are, it is difficult to be sure of their identity without cell sorting and examination. The images associated with the flow cytometric data reinforce this idea—different species do have characteristic light scattering/fluorescence signatures, but these generally form a continuum, and often overlap, and therefore are not very useful in determining species composition. The homogenous populations of cells indicated by the image groupings in FIG. 5 are not random selections, but were obtained by trial-and-error searches of small regions of the plot; other regions show mixtures of species. Thus, imaging allows greater improvement of the accuracy of the identification of different cells.

The ultimate resolution of the optical system is determined by the 10× microscope objective, which has a theoretical resolution of approximately 1 μm. As presently configured, an approximately 20 μm bead spans approximately 68 pixels (approximately 3.4 pixels/μm), so the camera resolution is more than adequate for this objective. However, image quality will be affected by several additional factors in the apparatus, including, particle motion, flash lamp pulse duration, and the location of particles in the flow cell.

Movement of the subject due to sheath flow during the camera exposure will tend to blur the image in the direction of flow. Sample particle velocity was determined (by measuring the image displacement caused by a known change in strobe delay) to be approximately 2.2 m/s, so the subject moves approximately 7.5 pixels during the approximately 1 μs exposure. The effect of this movement is visible in an image of a plastic bead as a thickening of the leading and trailing edges, relative to the upper and lower edges (not shown). In addition, although most of the light energy from the xenon flash is emitted within approximately 1 μs, the flash decays over several μs, which produces a “shadow” downstream of high-contrast subjects. These factors limit the velocity of flow that can be employed, and thus the sampling rate of the instrument. Although a shorter flash, as from an LED or pulsed laser, could be used to address this limitation. Accordingly, in yet another embodiment, the xenon flash is less than approximately 1 μs.

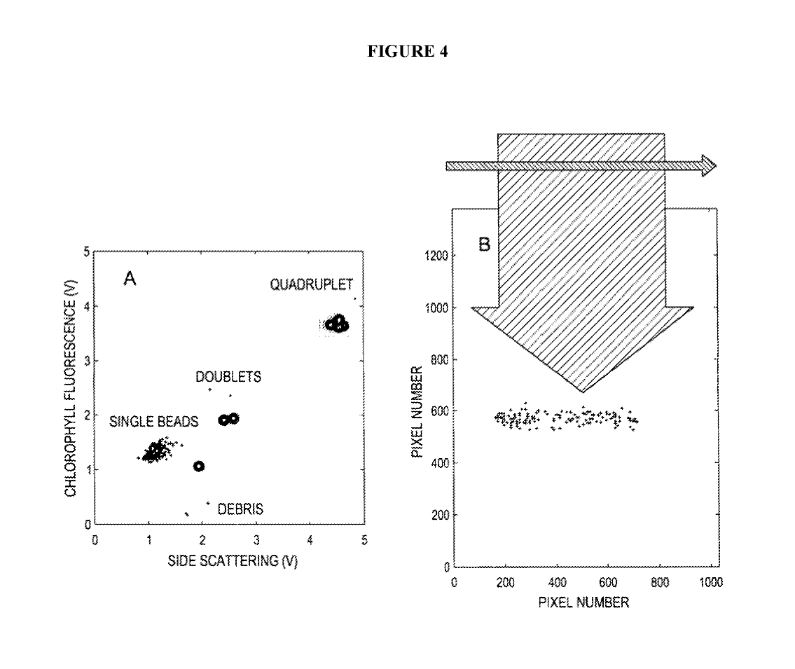

The sample core in the apparatus is approximately 150 μm wide (see FIG. 4B). If it is assumed that the core has the same shape as the channel, the thickness of the core would be approximately 33 μm. This is somewhat greater than the theoretical depth of focus of a 10× objective with N.A. 0.2 to approximately 10 μm. As the thickness of the sample core increases, more particles will be out of focus, which will limit both the sampling rate and the optical resolution that can be employed. Finally, the illumination conditions (e.g., condenser aperture, which is dictated by the amount of light available during the flash) affect the resolution and contrast of the image.

### Example 2

### Detection of Stained Phytoplankton

In one embodiment, the submersible flow imager provides automated, quantitative measurements of the abundance and properties of nano- and micro-phytoplankton by triggering its imaging system on chlorophyll autofluorescence as individual particles pass through a laser beam. Heterotrophic cells trigger detection if they have recently eaten a phytoplankton or have retained chloroplasts from phytoplankton prey, but heterotrophs lacking chlorophyll fluorescence are not well sampled by this instrument. Scattered laser light can be used as a trigger for all particles, but in practice this strategy is rarely useful because the large numbers of triggers from detrital particles decrease the efficiency of the system for detecting the rarer cells of interest.

In one embodiment, the submersible flow imager solves this issue by applying a stain that causes living cells to become fluorescent and therefore candidates for triggering the system. The submersible flow imager combines several necessary components, which are configured to fit within the submersible housing, and a programmed series of operations:

1) Fluorescence emission from the stained sample must be at a different wavelength from that of chlorophyll (which emits in the red, at 680 nm). Fluorescein Diacetate (FDA), which emits fluorescence in the green (530 nm) region of the spectrum was used. FDA is also convenient for use in an in situ instrument because, when dissolved in acetone, it is stable at room temperature. Other stains (e.g., LysoTracker Green®) can also be used.

2) The stain (in one embodiment, FDA) to be used for triggering must be excited with the same laser used for chlorophyll triggering, so the submersible flow imager's 635 nm laser was replaced with a 508 nm diode laser (Power Technology, Inc.). An added benefit of the 508 nm laser is that it excites fluorescence from phycoerythrin, as well as from chlorophyll, enabling discrimination of cells containing phycoerythrin (such as cryptophytes) from other phytoplankton.

3) A new photomultiplier was added to detect the new fluorescence signals.

Due to size constraints, the same photomultiplier is used for both stain fluorescence (green) and phycoerythrin fluorescence (orange). In alternating stained/unstained samples, optical filters are changed to isolate signals from green (stain) or orange (phycoerythrin) fluorescence. In a situation where space constraints are lifted (a laboratory instrument), an additional photomultiplier and filter could be added to measure green and orange fluorescence simultaneously.

4) The new signals are analyzed with an unused channel in the submersible flow imager's signal processing module.

5) Changing between optical filters for detecting green (stain) and orange (phycoerythrin) fluorescence is accomplished by a motor-operated cam and lever that slides a 2-place filter holder in the filter slot of the submersible flow imager's detection system module.

6) Staining is accomplished in a custom-built mixing/incubation chamber equipped with a magnetic stirring bar (operated by the submersible flow imager's bead-stirring motor). The chamber is connected to a spare port of the submersible flow imager's distribution valve; an approximately 5 mL water sample is aspirated into the submersible flow imager's syringe and pumped to the chamber for stain addition and incubation, then aspirated again and pumped through the flow cell for analysis and imaging.

7) Stain addition is accomplished by a solenoid-activated micropump (Reet Corp.) which injects a small volume (e.g., approximately 10 20 or 30 μL) of concentrated stain stock into each water sample before analysis by the submersible flow imager. The small volume is important to allow long term deployments with a practical volume of stain stock (i.e., approximately 6 months at approximately 36 samples/day requires approximately 130 ml stain stock).

8) In one embodiment, to avoid increased background fluorescence due to accumulation of fluorescent stain in the recirculating sheath, two solenoid valves are used to disable sheath recirculation during stained-sample analyses. At these times, external water is introduced to the sheath intake (through a pass-through in an unused plug in submersible flow imager's cap) and the flow cell outlet is directed overboard. During non-staining operations, the sheath fluid is recirculated to conserve filter capacity.

All functions for staining and analysis are under automated control through the submersible flow imager's electronic system and integrated processor and custom control software.

### Example 3

### Low Relief and Vehicle-Ready Embodiments

| | | | Preferred Configuration |
|---|---|---|---|
| | | Application | or Configuration |
| | Vehicle | Requirements | Requirements |
| 3.1 | Wave Glider | Accommodate very low | Ultra-low relief (≦17 cm) |
| | | vertical clearance in | with horizontal Pumping |
| | | payload bay | Subsystem |
| 3.2 | Autonomous | Accommodate wave- | Low relief (≦30 cm) with |
| | kayak | induced tilt/roll, | horizontally separated |
| | | moderate clearance | modules, reduced sample |
| | | payload bay | core width if needed |
| 3.3 | Raft | Moderate clearance | Low relief (≦30 cm) with |
| | | payload bay | horizontally separated |
| | | | modules |

Overview.

An autonomous vehicle-ready version of the instrument (IFCB-AV) that can be deployed on diverse vehicles enables high resolution plankton studies with both long duration and spatial coverage.

IFCB-AV is developed for two different autonomous vehicles to suit different research goals. Laboratory-based tests with a conventional IFCB oriented substantially horizontally (as required for deployment on small vehicles) guides design and operational modifications needed for long-duration deployments. An in-water (towed) deployment mode is preferred for compatibility with a wide range of vehicles and to simplify instrument temperature control and water sampling. In the second design, the instrument is redesigned to obtain vertical water flow through the critical analysis region but with modular layout of the remaining components. In this case, the instrument will fit within a flat box compatible with a range of vehicle hulls.

Because of the current gap in technology for observing phytoplankton at appropriate spatial scales, access to shared-use IFCB-AVs (with vehicles) will advance knowledge in a wide range of aquatic research areas. Documented research topics that will benefit from IFCB-AV include effects of climate change on plankton community structure in US coastal waters and the Arctic, causes of variability in shelf break ecosystems that support critical fisheries, dynamics, and regulating mechanisms of seasonal phytoplankton blooms, and early detection and understanding of HABs in the Gulf of Mexico and the Gulf of Maine.

The IFCB-AV is compatible with multiple existing autonomous oceanographic vehicles, well-suited for shared-use by a wide range of aquatic researchers. The IFCB-AV design will be readily manufacturable and thus can be made commercially available to the scientific community. Simply mounting an IFCB in the vertical orientation of its original design is impractical for almost any autonomous vehicle, due to size, stability, and center of gravity issues if hull-mounted, and to excessive drag if the instrument were to be towed. However, preliminary tests with an IFCB in our laboratory show that the instrument can also perform well in substantially horizontal attitude, so we propose to configure the instrument to operate on its side.

Instrument location is another important consideration. Within-hull mounting of an approximately 30 inch long IFCB is feasible for few autonomous vehicles, but almost any vehicle can tow an instrument (albeit at some cost in mobility). In addition to maximizing the pool of compatible vehicles, operating the instrument in the water simplifies temperature control and eliminates the need for transporting sample and waste water in and out of the vehicle hull. A further advantage is that an underwater instrument experiences less motion than one within the hull, which could enhance performance. It is also possible to sample at different depths with a towed instrument by adjusting tow angle; this could be controlled by adding a pressure (depth) sensor to the instrument and adjusting vehicle speed during intervals when the sample syringe is being filled. Such a strategy would also avoid sampling water affected by the propulsion jet in the case of motorized vehicles.

As researchers evaluate and refine predictions of change in the Arctic, it will be increasingly important to have spatially, temporally, and taxonomically resolved observations of important groups of plankton. While the ship-based efforts to date have provided new insights, the real advances will come from a combination of ships and multi-faceted autonomous sampling to avoid the pitfalls of limited ship access and of confounding space and time. An autonomous mobile IFCB is a critical element of future investigations of rapidly changing ecosystems near ice edges.

Design.

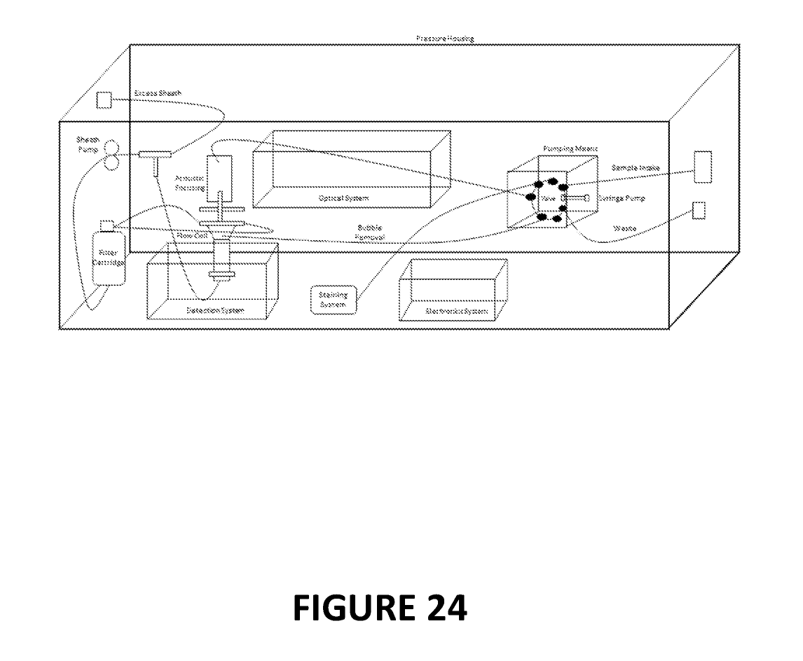

As shown in FIG. 24, the instrument layout may be redesigned so that the flow remains vertical though the analysis region. To accomplish this (without going back to the fully vertical instrument), the flow cell module is separated from the other components as to lay the modules out horizontally. The instrument would then have a low profile compatible with in-hull deployment. In such cases, a payload bay box may be employed for a commercial surface vehicle (Liquid Robotics Wave Glider SV3) and the Low Relief IFCB (IFCB-LR) to fit this box. The resulting IFCB-LR would be compatible with selected vehicles with relatively large in-hull payload areas.

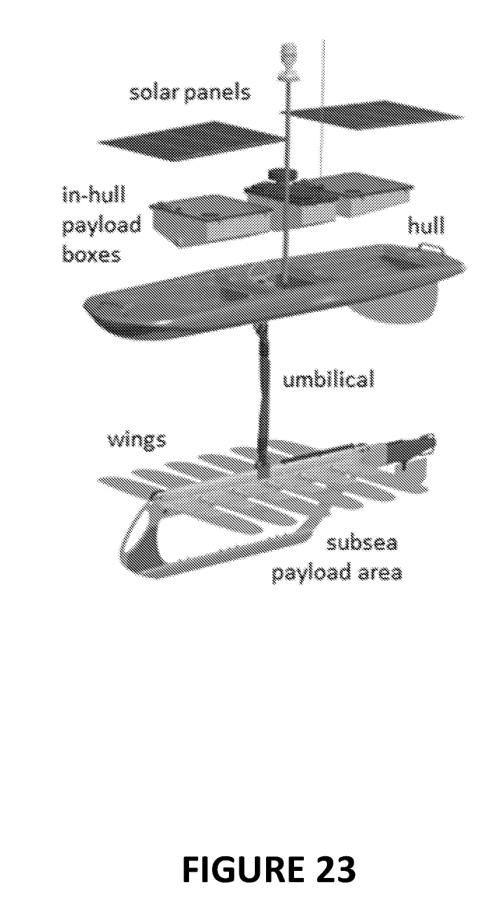

The need for plankton monitoring from autonomous vehicles has been established, but until recently autonomous vehicles did not have the payload or power capacities required for instruments such as IFCB. With the advent of systems such as Wave Glider and JetYak vehicles, these limitations no longer apply; the vehicle-ready IFCB can be deployed on such platforms and can be operated in diverse vehicles (FIG. 23). We will work specifically with a custom JetYak, to be constructed during the project, an SV2 Wave Glider, and, if appropriate, the more capable SV3 Wave Glider.

The design concept of IFCB-AV adheres to that of the proven IFCB, with modifications as needed for effective mobile operation. Elements that must be retained include 1) the combination of video and laser-based flow cytometric technology that make it possible to both capture images of nano- and microplankton and measure their chlorophyll fluorescence and light scattering; 2) subsystems for self-cleaning and analysis of on-board standards; and 3) the automation and control features that permit unattended deployment. The design relies on a precisely configured optical system and hydrodynamic focusing to produce highly controlled flow conditions and consistently focused images with resolution high enough (˜1 μm) that many plankton taxa can be recognized to genus or even species. To produce useful knowledge, IFCB data collection is coupled with a data and information system.

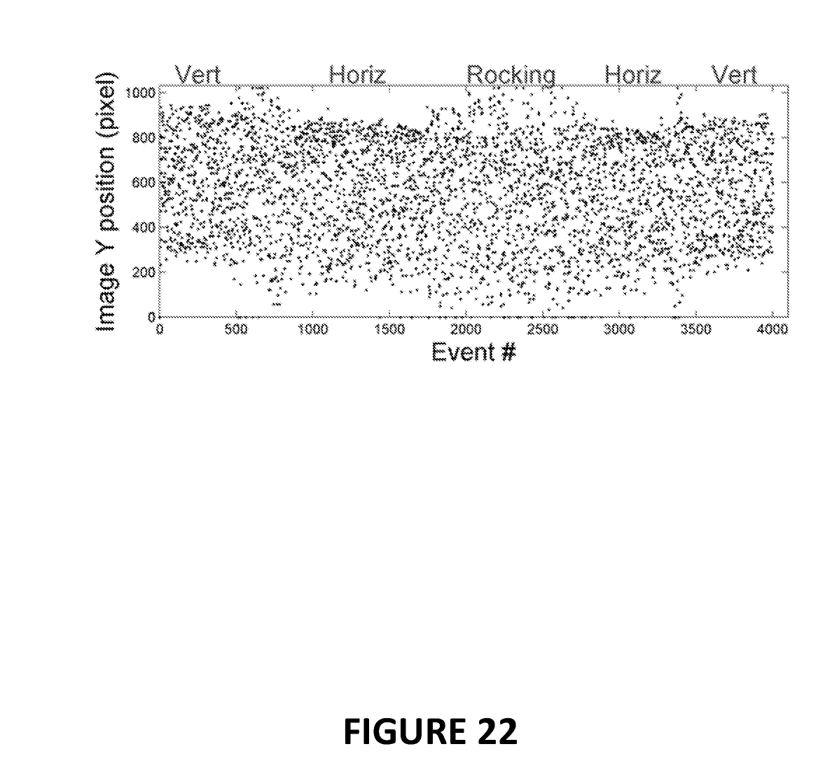

The first major design consideration for developing IFCB-AV is orientation. Simply mounting an IFCB in the vertical orientation of its original design is impractical for almost any autonomous vehicle, due to size and stability issues if hull-mounted, and to excessive drag if the instrument were to be towed. However, preliminary tests with an IFCB in our laboratory show that the instrument can also perform well in horizontal attitude (FIG. 22), so the simplest solution is configuring the instrument to operate on its side.

Instrument location is the next most important consideration. Within-hull mounting of a horizontal IFCB is feasible for few autonomous vehicles (one of the exceptions being a JetYak); the cargo bays of even the largest Liquid Robotics Wave Gliders are too short for the current 30 inch long pressure vessel. In contrast, almost any vehicle can tow an instrument (albeit at some cost in mobility). In addition to maximizing the pool of compatible vehicles, operating the instrument in the water will simplify temperature control (temperature changes can require refocusing) and eliminate the need for transporting sample and waste water in and out of the vehicle hull. A further advantage is that an underwater instrument should experience less motion than one within the hull, which could enhance performance. It is also possible to sample at different depths with a towed instrument by adjusting tow angle.

The current debubbling strategy is to periodically aspirate fluid from the conical chamber just prior to (above) the flow cell, where air bubbles accumulate when IFCB is vertical; when the system is horizontal this port may not be positioned well for removing air. The solution to this particular problem could be as simple as rotating the instrument so that the port (which is actually located in the side of the chamber near its “top”) can again aspirate the chamber effectively; or it may be necessary to identify a more effective location for this port, or to slightly tilt the instrument (or the pertinent components) away from horizontal.

One other potential problem with horizontal operation is that of particles settling out of a horizontal flow; such particles could aggregate and eventually cause flow blockage or disturbance. We expect that such problems will be prevented by the kind of periodic flushing with cleaning agents (bleach, detergent) which are now used to keep IFCB's sample tubing clean, but this will have to be tested during extended analyses of seawater samples from Woods Hole Harbor (as well as suspensions of known particles) followed by disassembly and inspection of critical areas of the system.

### Example 4

### Staining IFCB (IFCB-S). A standard IFCB is modified

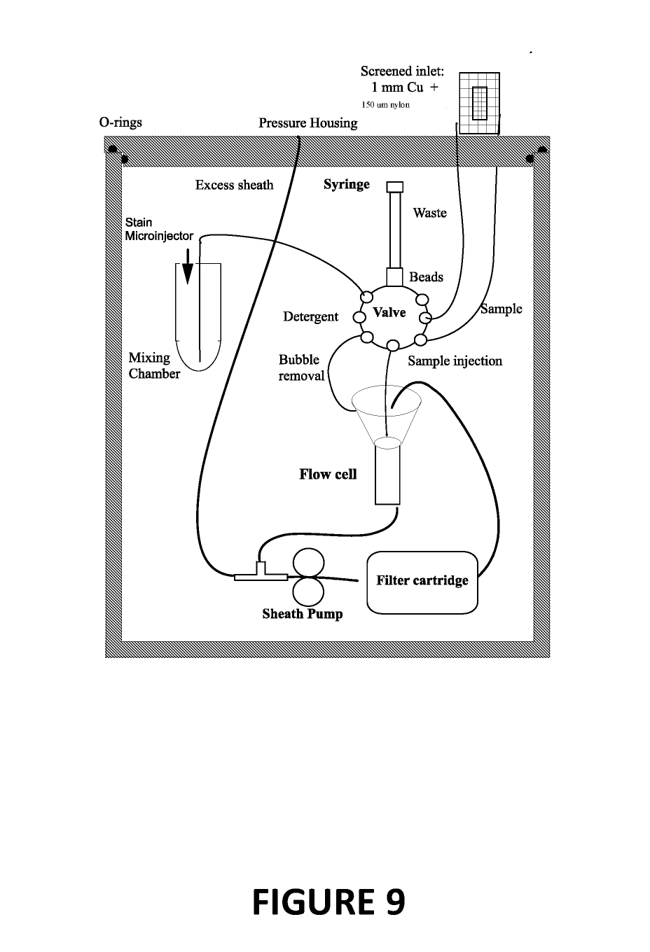

to carry out automated staining and incorporate optical components that enable it to detect either orange (as from phycoerythrin, PE) or green (stain) fluorescence, in addition to chlorophyll fluorescence. The optical and fluidic design for IFCB have been described in detail in Olson and Sosik (2007) A submersible imaging-in-flow instrument to analyze nano- and microplankton: Imaging Flow Cytobot. Limnol. Oceanogr.: Methods 5, 195-203. A sample (typically 5 ml) is drawn into the instrument by a programmable syringe pump. The sample water is injected into the center of a particle-free sheath flow in the cone above a rectangular quartz flow cell. In the standard IFCB, seawater is drawn into a sample syringe and then injected directly into the cone through a needle; sheath fluid is recirculated after passage through cartridge filters. For Staining IFCB (IFCB-S), a new fluidics control is added with features utilizing IFCB's distribution valve and new solenoid valves to allow for automated addition of stain, as well as for discarding sheath fluid during stained sample analysis (to prevent accumulation of stain in the system). Staining is carried out in a mixing chamber connected to an extra port on the valve. First, a microinjector (120SP2420-4EE, Bio-Chem Valve) adds 20 μl of concentrated stain to the empty chamber. Then, the seawater sample is pushed through the distribution valve into the mixing chamber, where it mixes with the stain and incubates (typically for 30 seconds) before being pulled back into the sample syringe and sent through the flow cell for analysis (FIG. 9).

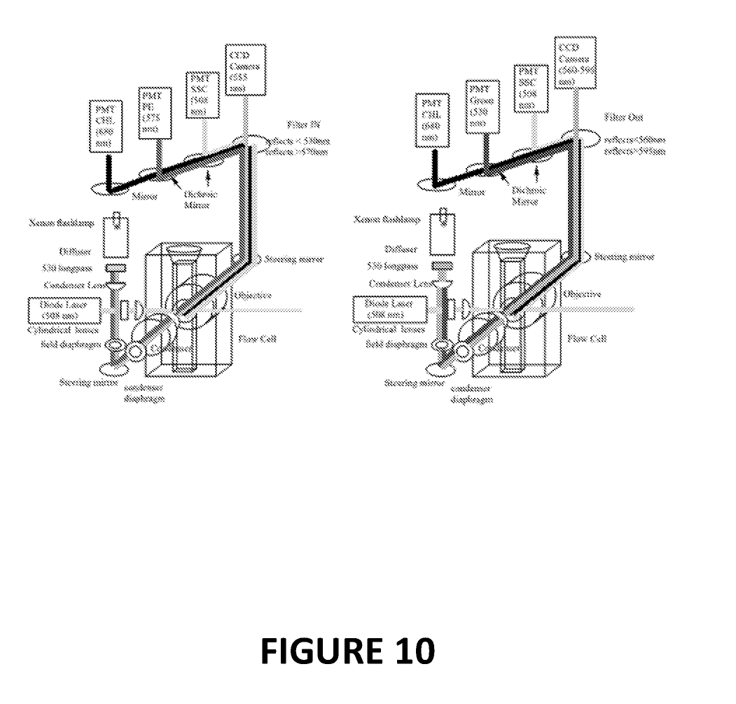

The IFCB excites chlorophyll fluorescence with a 635 nm diode laser. As a particle passes through the focused laser, laser light is scattered and chlorophyll-containing cells emit red fluorescence (680 nm). One (or more) of these signals, usually chlorophyll fluorescence, is used to trigger a 1 μs pulse from a xenon flash lamp. The green component of the lamplight is isolated by a bandpass filter and used for the camera exposure. Dichroic mirrors are used to separate the wavelengths used to detect chlorophyll fluorescence and side scattering (680 nm and 635 nm, respectively). In the modified optics for IFCB-S(FIG. 10), the 635 nm laser is replaced by a 508 nm diode laser (Power Technology, Inc., model PM20 (510-50) G4, 20 mW) that can excite fluorescence from the stain (530 nm), as well as from chlorophyll (680 nm) and phycoerythrin (575 nm). A 488 nm laser can also be used for this set-up, though it utilizes more power than the 508 nm laser. In this case, a 570 nm shortpass filter must be inserted before the photomultiplier tube that detects PE as a laser using 488 nm can cause Raman scattering, which emits at wavelengths similar to PE. We incorporated an automated optical filter slider making it possible to detect either orange (PE) fluorescence for unstained samples or green (FDA) fluorescence for stained samples. To detect FDA fluorescence, IFCB-S uses a “double dichroic” (Omega Optical, 595 DMSP), which transmits light between 560 and 595 nm to the camera and reflects light below and above this band to the photomultiplier tubes. To detect PE fluorescence (when samples are not stained), IFCB-S uses a 555 DMSP, which transmits 530 to 570 nm and reflects longer wavelengths.

Staining Validation—

A cultured marine bacterivorous scuticociliate (Uronema marinum, isolated from Buzzards Bay, Mass. in 1986) was used to evaluate initial IFCB-S performance. Cultures were maintained at 15° C. on a 14:10 h Light:Dark cycle and transferred weekly into 40 ml sterile filtered seawater with 1 drop yeast extract and 2 rice grains. As a control, scuticociliate cells were imaged with IFCB-S triggering on scattering to ensure detection of all cells. To evaluate effective stain detection, cells were then analyzed with IFCB-S triggering only on green fluorescence with and without stain added.

Stain Comparison—

To compare detection efficiency between LTG and FDA, scuticociliate cultures were sampled daily during batch growth, stained by either LTG or FDA, and cell counts were determined with a FACSCalibur™ flow cytometer detecting only green fluorescence.

Comparison with Conventional Microscopy—

Environmental samples were collected from Woods Hole Harbor. Live samples were pre-filtered through a 250 μm mesh and kept under conditions of in situ temperature until analysis on IFCB-S. Samples were run on IFCB-S in staining and non-staining modes (50 ml of sample run for each mode). A 200 ml sample was fixed with 10 ml acid Lugol's solution (final concentration 5%, modified from Throndsen 1978). Fifty-ml acid Lugol's-fixed samples were settled for 24 h in Utermöhl chambers, and cells were subsequently enumerated under an Axiovert S100 inverted microscope at 40× magnification.

Microzooplankton counts from manual light microscopy were compared to those from IFCB-S in staining mode and IFCB-S in non-staining mode. For these comparisons, ciliates were grouped into four taxonomic categories: tintinnids, Mesodinium sp., Laboea strobila, and “other ciliate taxa”. The heterotrophic dinoflagellates, Gyrodinium sp. and Protoperidinium sp. were also considered for comparison. Analyses were performed during three seasons; winter, spring, and summer (with the winter sample lacking manual light microscopy). Poisson distribution statistics were used to calculate 95% confidence intervals for counts. The E-Test statistic described by Krishnamoorthy and Thomson (2004) was used to test for significant differences.

Comparison of Detection Between IFCB and IFCB-S—

For field assessment, IFCB-S was used during the National Maine Fisheries Service Summer Ecosystem Monitoring Survey (ECOMON, EX-13-05) aboard the NOAA Ship Okeanos Explorer. The cruise track covered the continental shelf from southern New England waters northward through Georges Bank and the Gulf of Maine to Nova Scotia Shelf waters. IFCB-S was used side-by-side with a standard IFCB for continuous sampling of water from the ship's underway system (3 m sample depth). The standard IFCB triggered on chlorophyll fluorescence, while IFCB-S was configured to run alternate sample modes: one staining, with triggering on chlorophyll and/or stain fluorescence and, the other non-staining, with triggering on chlorophyll and/or PE fluorescence.

Automated Classification of a Time Series—

Routine analysis of IFCB data includes image processing, feature extraction, and supervised automated classification, except that instead of the original support vector machine, we now use a random forest classification algorithm after Breiman (2001). We applied a classifier with 50 categories, including Laboea strobila, mixed tintinnids, and mixed other ciliates. For each unknown image, results from the classification algorithm (TreeBagger function in MATLAB, The Mathworks, Inc.) provide an affiliation score for each class (scores sum to 1 across all classes). By selecting a score threshold above which classifications are accepted, it is possible to reduce the incidence of false positives, albeit typically at the expense of lower probability of detection for true positives. The efficacy of this approach is demonstrated here by comparing intermittent manual image identification with a high-resolution multi-year time series of cell abundance from the automated classifier for the ciliate species Laboea strobila at MVCO. Regression analyses between manual and automated counts for various score thresholds were performed and R² values, y-intercepts of the lines of best fit, and slopes of the lines of best fit were used to evaluate the best score threshold. An ideal threshold would be chosen where the r² was maximized, the y-intercept was near zero, and the slope approaches 1.

Results.

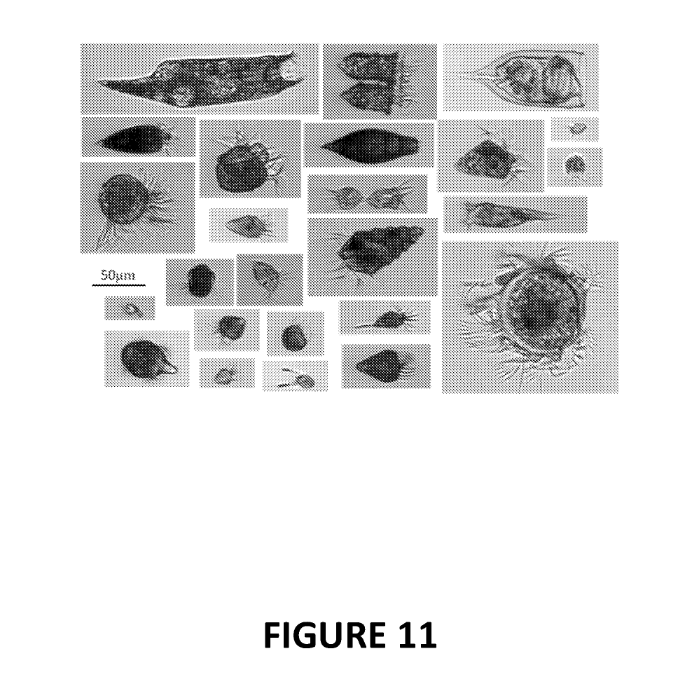

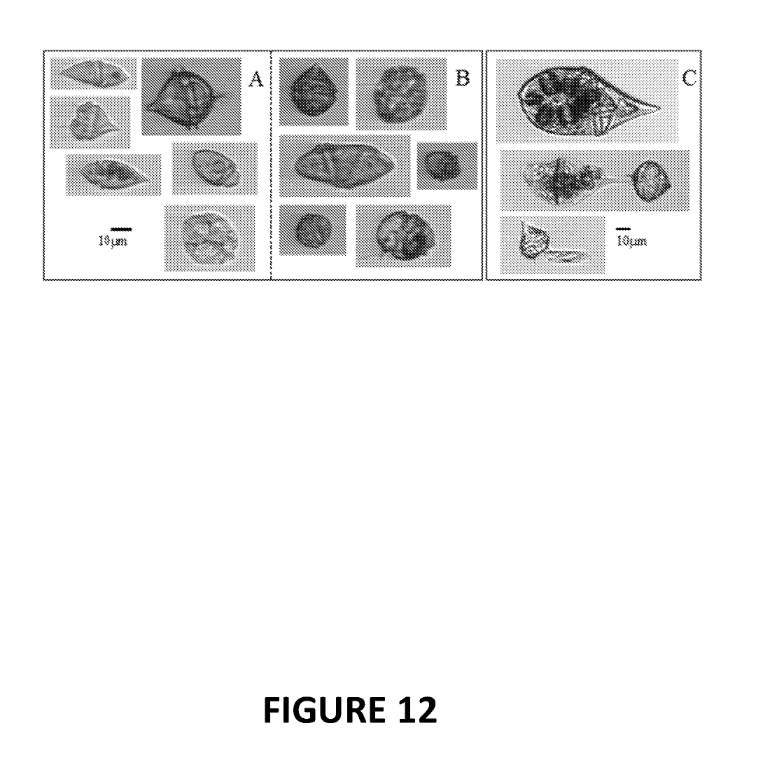

At MVCO, the predominant ciliates detected by the standard IFCB come from the Spirotrichea subclasses Oligotrichia and Choreotrichia (FIG. 11). The photosynthetic ciliate Mesodinium sp is also readily detected due to its mixotrophic nature. More rare ciliate taxa include the haptorid, Didinium sp and the prostomatid, Tiarina fusus. While identifying ciliates down to species with image resolution provided by IFCB (1 μm) may be difficult, they can be grouped by similar morphology and functional group inferred from previous literature. Distinctive taxa, such as Laboea strobila, can be identified to the species level. Heterotrophic dinoflagellates are also detected if they are consuming phytoplankton (FIG. 12). These are predominantly gyrodinoid and gymnoid forms. Occasionally Protoperidinium sp. and Amphidinium sp. are observed.

Performance of IFCB-S—

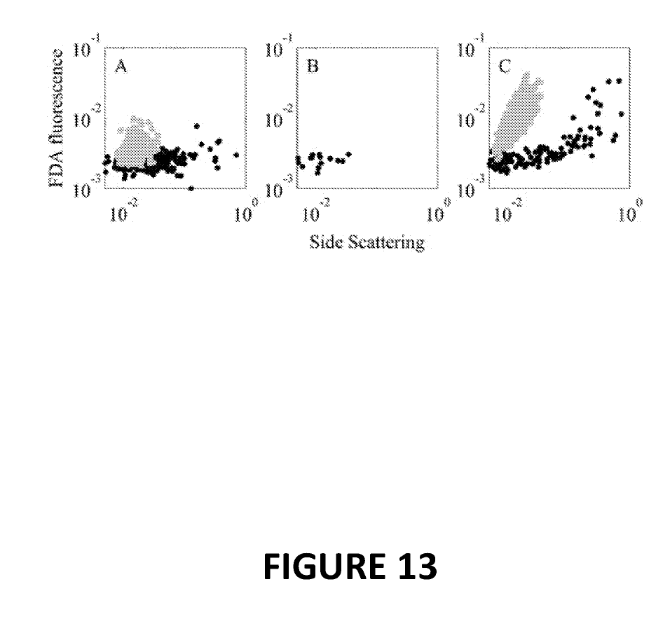

To evaluate IFCB-S's ability to stain and detect ciliates lacking chlorophyll fluorescence, a bacterivorous scuticociliate culture was used. On a traditional IFCB triggering on chlorophyll fluorescence, these ciliates would not trigger image capture. Initially, we used a side scattering trigger to detect all particles (FIG. 13A). In this case, both detrital particles and ciliates were imaged, with detrital particles dominating but ciliates readily detectable. When a non-stained cell culture was analyzed on IFCB-S configured to trigger on green fluorescence, no scuticociliates were detected, as expected since these cells are not highly autofluorescent (FIG. 13B). Once cells were stained, they were readily detected with a green fluorescence trigger (FIG. 13C). Triggering on stain fluorescence ensures that more time is spent imaging the ciliates (instead of detritus). Use of a scattering trigger or fluorescence trigger allowed a similar volume to be analyzed during a 20 minute period (2.26 ml at an average trigger rate of 1.12 particles s⁻¹, 2.27 ml at an average trigger rate of 1.08 particles s⁻¹, respectively), but in the case of staining and fluorescence triggering, 75% of the images contained ciliates compared to only 41% with scattering.

Comparisons of Stain—

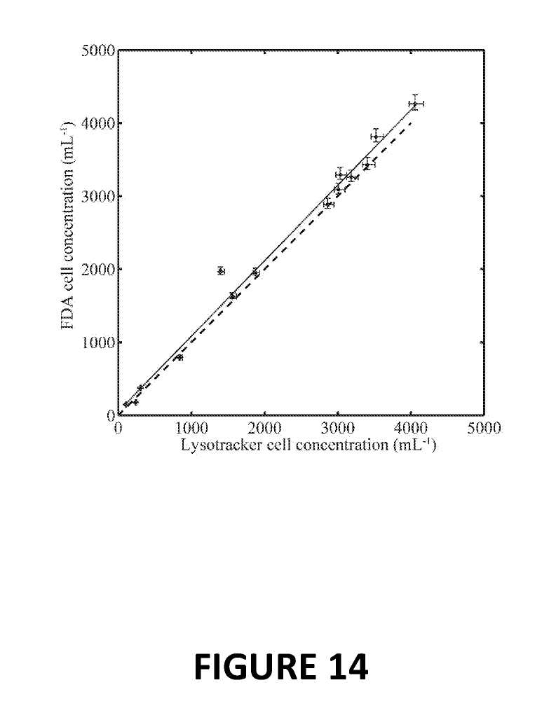

To compare the performance of LTG and FDA with new groups, scuticociliate cell counts were determined by conventional flow cytometry triggering on green fluorescence. Detection efficiency was similar between the two stains (FIG. 14), allowing for further considerations to be used in selecting the optimal stain for use in IFCB-S. Further application of FDA was selected due to its stability in solution for up to 6 months at room temperature (personal observation), as well as its lower cost. Recommended storage for LTG is −5 to −30° C. which presents challenges for long term in situ deployments.

Comparison of IFCB-S and Manual Microscopy—

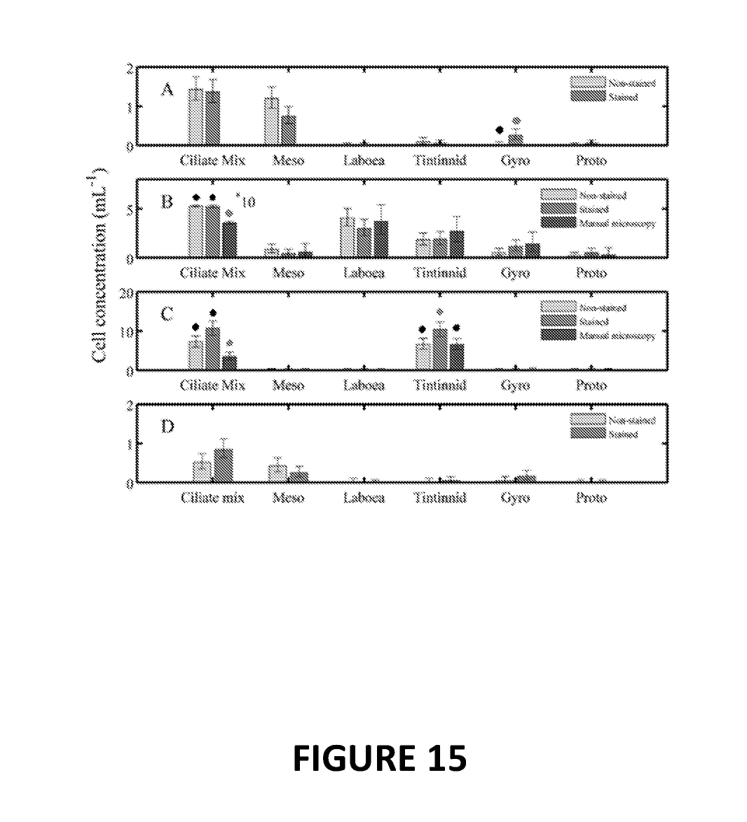

Performance of IFCB-S on environmental samples was evaluated by comparison with conventional modes of counting protozoa: settling and manually counting cells in acid Lugol's stained samples. IFCB-S counts were also compared with and without staining. Specifically, abundances for Mesodinium sp., Laboea strobila, mixed tintinnids, Protoperidinium sp., and mixed gyrodinoid dinoflagellates were compared. During a comparison of wintertime samples, no significant differences were found between ciliate morphotypes detected by IFCB-S with and without staining (FIG. 15A). In stained samples, however, higher abundances of a gyrodinoid dinoflagellate was detected, indicating these organisms are likely consuming heterotrophs and thus often missed by standard IFCB with a chlorophyll trigger (FIG. 15A). During a springtime comparison, IFCB-S detected approximately 25% more mixed ciliates than microscopic analysis (FIG. 15B). There were no significant differences in abundances for other micrograzer morphotypes between the methods. There were also no differences in detection between staining and non-staining modes, consistent with most protists contained chlorophyll either in their guts or in retained plastids. A summertime comparison allowed only for comparison in the ciliate mix and tintinnid groups as other types were consistently undetectable (FIG. 15C). For the detected ciliate types, both stained and unstained sample concentrations were significantly higher than manual microscopy. A fall comparison did not show any significant differences between staining and non-staining modes (FIG. 15D).

IFCB-S Field Application—

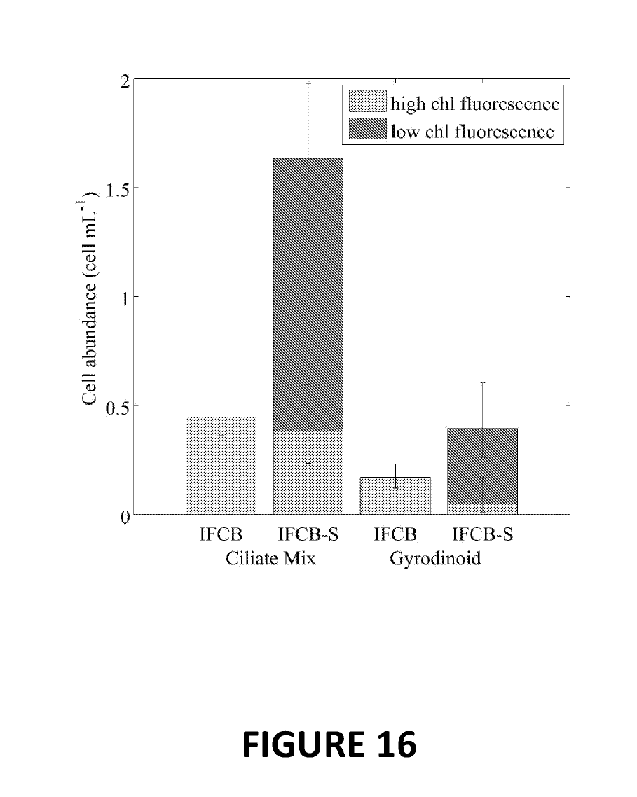

IFCB-S was configured for automated underway sampling of surface waters during a cruise over the northeast US continental shelf. We examined ciliate diversity and abundance and compared morphotypes that did and did not ingest algae. Two populations of organisms were observed in the stained samples: one with high red fluorescence and one with little to no red fluorescence; both showed a range of green fluorescence that roughly corresponded to cell size. Ciliates were present in both of these groups, so it was possible to detect a greater number of total ciliates in stained samples. This was due to taxa present in the low red/high green fluorescence population (FIG. 16).

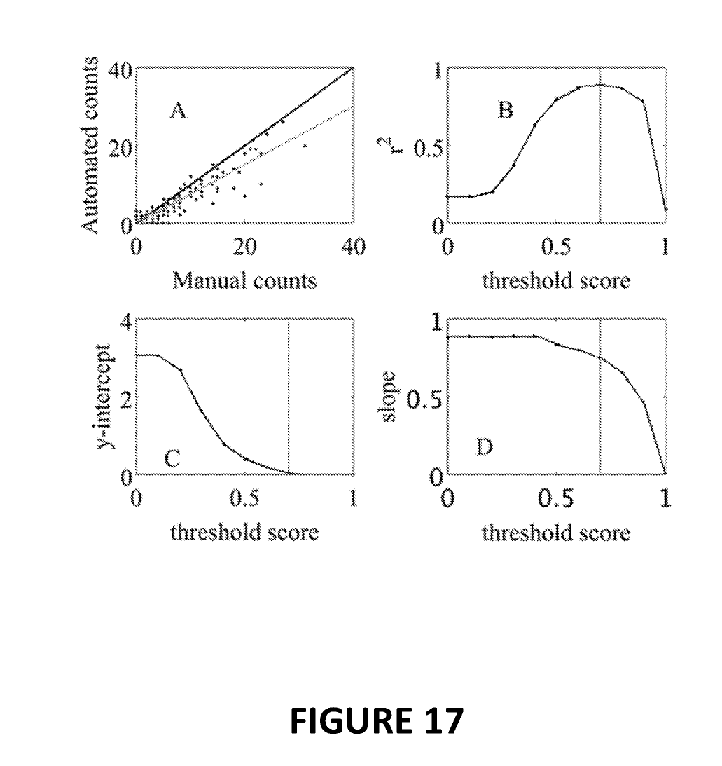

Observations of tintinnids during the cruise provided a notable example of the advantages of IFCB-S. We found two groups of tintinnids in the stained samples: one with high red fluorescence and one with little to no red fluorescence; as expected, both groups exhibited green fluorescence. Only red fluorescent tintinnids were detected in the non-stained samples, with maximum concentrations reaching approximately 0.4 cells ml⁻¹. This population was captured by IFCB-S in similar concentrations, but the second population with little red fluorescence was detected only by this instrument with higher total tintinnid maximum abundances (˜1 cell ml⁻¹) (FIG. 17). The staining of samples consistently allowed for detection of a group of tintinnids that otherwise would not have been observed.

Automated Classification—

Automated classification is essential for analyzing the large sets of data produced by IFCB and IFCB-S. To do so, an optimal score threshold for automated classification was determined through a regression analysis between manual and automated classification results (FIG. 17). In the case of 0.7, the r² value was maximized, but the y-intercept was just above 0 and the slope was just below 1. This reflects the tradeoff between detection efficiency and low false positives.

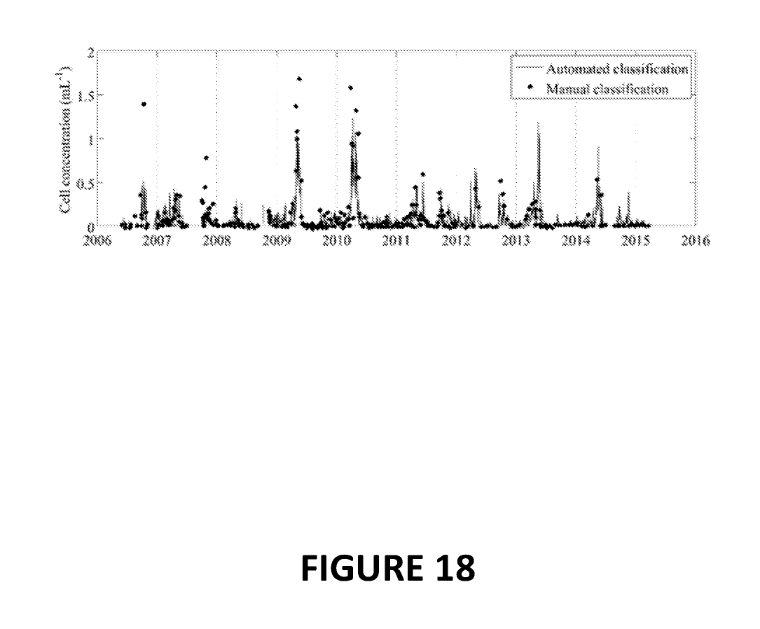

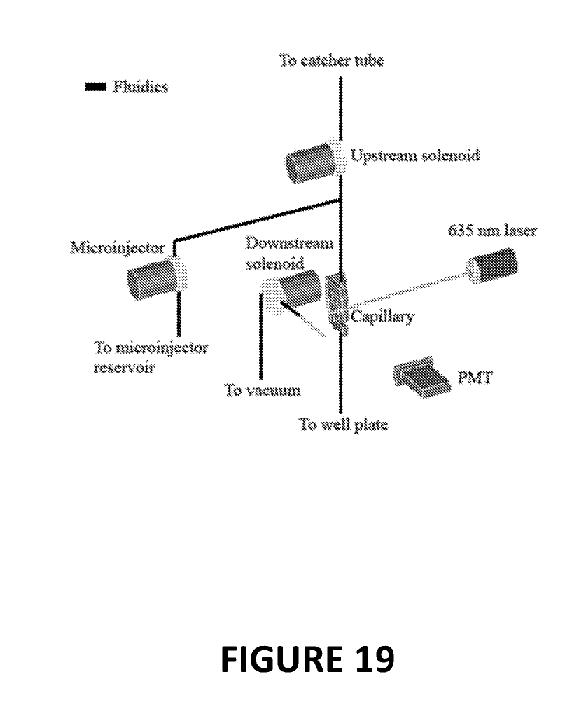

The efficacy and value of this approach was demonstrated for ciliates by comparing manual and automated identification of images for times series data for the ciliate, Laboea strobila (FIG. 18). In this case, a classifier score threshold of 0.70 was found that (images classified as L. strobila only if score associated with the class >0.7) provided acceptable automated classification results. Possible discrepancies between automated and manual identification may be the result of patchiness at MVCO and how the data is binned. In some cases, manual classification was only completed for a few hours within a given day, while the daily bin for automated classification encompasses every sample taken that day. If different water masses were moving by the MVCO offshore tower throughout the day, high frequency variability in cell concentration might lead to mismatches with the daily average values. Seasonal spring blooms of Laboea strobila occurred during April-May in most years, while fall blooms were smaller and more variable in timing (FIG. 19).

Discussion.

Protist micrograzers are key players in aquatic ecosystems yet they are difficult to study due to methodological challenges. The standard IFCB is a powerful tool for studying these organisms in situ. Because IFCB can be deployed long-term, it is effective for characterizing protozoan community structure with high temporal resolution. It can image a wide variety of grazers and provide insight into which organisms are present (e.g., FIGS. 11, 12), as well as their seasonal dynamics (FIG. 18). There are limitations, though, because the reliance on chlorophyll fluorescence for image triggering means standard IFCB is only able to quantify patterns of herbivores and mixotrophs. The addition of live cell staining is appropriate to take this observational technique forward to view a more complete community.

In typical cytometric analyses, there can be difficulty when discriminating a phototroph with concentrated stain from an herbivorous or mixotrophic protozoan because both can have high levels of chlorophyll fluorescence. Imaging technology allows to differentiate the two from the images associated with each cell. On the other hand, some grazers may have undetectable levels of chlorophyll fluorescence or none at all (for instance, those grazing on heterotrophs) and the addition of stain is necessary for efficient detection. There are a number of possible fluorescent stains that can be used to differentiate heterotrophs with flow cytometry. Several factors were considered in selecting a stain for use with IFCB-S, including whether the stain fluorescence can be differentiated from chlorophyll and can remain stable at ambient temperatures (important for long term in situ deployments). Most importantly, the wavelength of the laser must be able to induce fluorescence by the stain, but limit overlap of emitted wavelengths with scattered laser light. This criterion led to focus on LysoTracker® Green (LTG) and fluorescein diacetate (FDA) as candidates. Ultimately the use of FDA is recommended for extended in situ staining application. Its effectiveness is comparable to LysoTracker® Green (LTG) (FIG. 14), while its ability to remain stable at ambient temperatures and its affordability make it preferable. Because LTG stains the acid vacuoles created during digestion, it might be useful to distinguish those protists that are actively grazing, but personal observations showed general staining of all cells including pure autotrophs and not in relation to levels of grazing. Since chloroplasts can be acidic, some phototrophs can exhibit LTG fluorescence after staining. These may be recognized from their high stain-to-chlorophyll ratios, combined with cell characteristics evident in the images. With controlled analysis of a bacterivorous scuticociliate culture, automated staining was shown to be capable to readily detect and image grazers previously undetectable with IFCB (FIG. 13). While the amount of staining may be variable for different grazers, these results suggest that widespread detection of grazers without chlorophyll fluorescence is possible.

To test the effectiveness of protozoa detection by automated imaging in mixed assemblage natural samples, results were compared to those from manual light microscopy. For samples collected from Woods Hole Harbor in spring and summer, significantly higher abundances of mixed ciliates were detected with IFCB-S compared to manual microscopy (FIGS. 15B, 15C). This suggests traditional counting methods involving preservation and settling may be detrimental to the cells. This is consistent with the conclusions of Stoecker et al. (1994) that no single method of fixation is ideal for all purposes, so taxon-and fixation-specific correction factors may need to be applied. Because the IFCB is used to image ciliates in situ without fixation steps, loss of delicate cells may be minimized.

The comparisons between staining and non-staining modes with IFCB emphasized the value added by combining automated staining with imaging. During summertime sampling in Woods Hole Harbor significantly higher counts of tintinnids and mixed other ciliates were observed in stained samples (FIG. 15C). These higher counts indicate many ciliates exhibited no chlorophyll fluorescence (or too little to measure with IFCB), so staining was required to detect them. This comparison also provides insight into aspects of feeding strategy: the ciliates only detected after staining are presumably not mixotrophs and were either not actively grazing or were grazing on heterotrophs. Various types of tintinnids are known to be heterotrophic so this result is not surprising for that group. Interestingly, we found no difference for mixed ciliates during the summer and for a spring sample comparison, we found no significant differences between staining and non-staining modes of the IFCB for any category. This likely indicates chlorophyll-containing micrograzers dominated, presumably a combination of mixotrophs and organisms actively feeding on autotrophs. Also working in waters near Woods Hole, Stoecker et al. (1989) similarly found that, during summer seasons when there is low phytoplankton biomass, autotrophic and mixotrophic ciliates can contribute high amounts of production, becoming important food sources for higher trophic levels. During winter sampling, a similar result was found for ciliates, but at that time a heterotrophic gyrodinoid dinoflagellate was much more abundant in stained samples (FIG. 15A). While taxon-specific differential feeding has been observed in both ciliates and dinoflagellates, seasonal patterns of this have not been heavily explored. The results suggest there could be taxon-specific differences in feeding strategies that vary with season.

Preliminary field applications of IFCB-S during the summer Ecosystem Monitoring Survey further demonstrate and support expanded capabilities to detect heterotrophic protists. The use of stain was found to allow for imaging of greater numbers of ciliates on the cruise transect by IFCB-S compared with a standard IFCB (FIG. 16). The additional cells detected by IFCB-S exhibited high ratios of green fluorescence to chlorophyll fluorescence, indicating these grazers were unlikely to have been ingesting algae. Some ciliate morphotypes were similar in abundance during staining and non-staining modes and exhibited a range of chlorophyll fluorescence. This may suggest low chlorophyll containing morphotypes were herbivorous but exhibited levels too small to detect by chlorophyll fluorescence alone and were thus only imaged when stained.

The use of stain also made it possible to detect additional ciliates during underway sampling on the cruise (FIG. 17). Significantly higher numbers of the tintinnid, Eutintinnus sp. were found than captured by the standard IFCB. Most of this population did not have chlorophyll fluorescence above the trigger threshold so they were not counted without stain. At the same time, a different group of tintinnids with agglomerated loricas, Tintinnopsis sp., were observed with both the standard IFCB and IFCB-S at similar abundances due to their consistently high chlorophyll fluorescence.

Taken together, these comparisons not only support the efficacy of automated staining, they also provide insight into the feeding modes of micrograzers. If similar morphotypes exhibit a range of high and low chlorophyll fluorescence, we can infer that all feed on autotrophs, but that those with consistently low levels of chlorophyll fluorescence relative to their size and stain fluorescence supplement their diets with heterotrophs. Morphotypes that consistently exhibit undetectable chlorophyll fluorescence are likely to be grazing predominantly on other heterotrophs. A single morphotype could be comprised of genetically distinct populations, possibly exhibiting different feeding strategies, in which case this would be reflected in a range of chlorophyll relative to stain fluorescence.

These kinds of analyses also prompt interesting questions about whether certain morphotypes exhibit different feeding modes through time (perhaps depending on prey availability). For example, we detected similar gyrodinoid dinoflagellate morphotypes throughout the year in Woods Hole Harbor, but whether they were dominantly chlorophyll containing or not differed with time (FIG. 15A, 15B, 15C). This observation is consistent with certain feeding strategies being more favorable than others at different times of the year, but more extensive observations are needed to determine if recurrent patterns occur seasonally. Heterotrophic dinoflagellates, such as Gyrodinium sp., have been observed to feed on a wide range of prey types, from pure autotrophs to other heterotrophic organisms such as bacteria and small flagellates. Though they have been observed to be dominant grazers on diatoms, this may not be the case in waters near Woods Hole. During the winter, when long chain diatoms dominate the autotroph biomass, most gyrodinoid dinoflagellates were not chlorophyll containing (FIGS. 15A, 15B, and 15C), indicating it may be more favorable for them to feed on smaller heterotrophs. It has been noted that heterotrophic dinoflagellates may at times outcompete other grazers by being able to efficiently maintain metabolism at low prey concentrations. A possible interpretation of the results is that these dinoflagellates are feeding less in the winter. During the spring and summer, when the gyrodinoid morphotype was predominantly chlorophyll containing, it may have been feeding on small autotrophs. Certain species of gyrodinoids (Gyrodinium dominans), for instance, have been found to respond quickly to increases in cryptophytes, which can be important at that time of year. Interestingly, observed was a contrasting pattern for ciliates, which appear to be predominantly herbivorous or mixotrophic during the winter (FIG. 15A). These ciliates seem to be predominantly consuming autotrophs at the times when gyrodinoid dinoflagellates were not. This is perhaps surprising since the ability of the two to ingest autotrophs has been shown to be comparable. Perhaps this difference reflects ciliates having the potential to grow faster than their heterotrophic dinoflagellate competitors. In winter the dinoflagellates may be occupying a different niche associated with consumption of small heterotrophs. Though these analyses are only snapshots in time, they provide interesting insights that argue for studies of longer time periods to address questions of seasonality in a more quantitative manner.

Addressing these types of questions with large image data sets that include this more complete community of heterotrophs raises immediate data analysis challenges, and automated image analysis and classification will be imperative. We can build from the approaches used for phytoplankton (Sosik & Olson 2007) to develop automated classification for these new populations. While work remains to extend automated classification to a wide range of protist morphotypes, we have shown efficacy for selected ciliates. For Laboea strobila, in particular, we can detect recurrent blooms and seasonal patterns with automated classification, as verified by intermittent manual identification of images (FIG. 17). Our analysis emphasizes a recurrent spring bloom (FIG. 18), which is consistent with seasonal trends documented for Laboea strobila in the Gulf of Maine also observed a strong spring peak in the abundance of this species during a three year study in the Mediterranean Sea. It remains to be determined what factors drive the similar spring increase between both New England and other temperate waters. Interestingly, the high resolution time series has uncovered an additional more variable and smaller amplitude fall increase in Laboea abundance (FIG. 17). Whether this is a feature in other systems is currently unknown.

We have demonstrated that the expanded observational capabilities of IFCB-S make it possible to use live cell stains such as FDA to uncover a more complete micrograzer community in natural waters. When coupled with automated image analysis and classification this allows us to explore the diversity, dynamics, and ecosystem roles of protistan grazers in new ways. Not only are we now able to detect populations grazing on heterotrophs (those with undetectable chlorophyll fluorescence), but we have shown that some taxa can now be detected at higher abundances than observed with traditional manual light microscopy coupled with settling of preserved cells. Because IFCB-S requires little sample handling and no preservation, it likely has reduced loss of delicate cells. Furthermore, continuous, high temporal resolution sampling has important advantages. Long duration time series permit detection of more rare species of grazers likely to be missed in intermittent small volume samples. Spatially resolved sampling, such as the underway cruise sampling described here, emphasize that both standard IFCB and IFCB-S can to detect ciliate ‘hot spots’. Station-based sampling on the same cruise provided far lower spatial resolution, with the result that patches would have been difficult to detect and characterize. We also have the power to resolve feeding habit and its possible plasticity, for instance as seen in seasonal changes in whether certain morphotypes exhibit chlorophyll fluorescence from retained chloroplasts or undigested autotrophic prey.

After a review of published data on microzooplankton grazing, Schmoker et al. (2013) mention the need for more time series and higher taxonomic resolution during grazing studies. Though long-term data sets of protist micrograzers are not common, a few studies have highlighted the power of studying systems over long periods of time. Modigh (2001) observed similar patterns of succession in ciliate taxa every year for three years, possibly indicating reduced competition between taxa and a diversified grazing pressure. During a year-long study, Lawrence & Menden-Deuer (2012) found seasonal changes in grazing rates corresponded more to prey community composition than temperature. The IFCB-S allows for the much needed long term studies of microzooplankton communities in situ and through high resolution images provides further exploration of diversity. Because the IFCB-S also samples phytoplankton communities, going forward we expect this observational technology to enable unprecedented exploration of predator-prey interactions and patterns through space and time.

### Example 5

### IFCB-Sorter

Flow cytometry has deep roots in marine ecology and has contributed immensely to our understanding of the ecology and biogeography of the world's oceans. Perhaps the most striking discovery that can be largely attributed to the application of flow cytometry to marine microbial ecology is that of the existence of Prochlorococcus. Additionally, large contributions to our understanding of global distributions of marine phytoplankton are largely due to surveys carried out using flow cytometry as the main technology. In present day, flow cytometers are still an essential element in our understanding of plankton spatiotemporal diversity. Along with a greater understanding of ocean biogeography and biodiversity, flow cytometry was also becoming a well-developed tool for studies concerning the cell cycle and physiology of marine planktonic organisms.

More recently, fluorescence activated cell sorting (FACS) have been used in combination with variety of chemical analyses to better understand biogeochemical cycling to delve into interactions between bacteria and phytoplankton and to investigate intra-species genetic variability. These studies have highlighted the incredible heterogeneity at the single-cell level. With incredible leaps in single-cell genomics and transcriptomics, cell-sorting flow cytometers will be of immense utility in isolating cells from the environment.

The instrument described here is a modified Imaging FlowCytobot, which operates as a benchtop instrument capable of sorting individual cells. The Imaging FlowCytobot is a submersible imaging-in-flow cytometer, which can be used to characterize planktonic cells in the size range of 5-100 μm; it stores an image of the cell captured, and records the chlorophyll fluorescence and side light scattering of the cell. The instrument described here is in essence a fluorescence-activated cell sorting (FACS) system with several notable differences. Currently, fluorescence-activated cell sorters process anything satisfying predefined measurement criteria (typically, windows of fluorescence various wavelengths and of laser light scattering). This method of sorting often cannot distinguish between different species of organisms in the sample, so that it is necessary to investigate community composition by sorting many individuals more or less at random; species identity is then determined by analyzing (e.g., sequencing) each sorted cell. The IFCB-Sorter, in contrast, captures an image of a micro- and nanoplankton cell or colony and then sorts it into a well plate. Since the images can in many cases be used to automatically (or manually) classify the cell to genus or even species, only the sorted cells of interest need by further analyzed. Additionally, examination of the image associated with the sorted cell can contain valuable information about the condition of the cell at the time of capture. Images acquired allow interesting questions on the relationship between morphology and genotype to be pursued.

The IFCB-Sorter works in a manner similar to both FlowCytobot and the Imaging FlowCytobot, in which a seawater sample is taken up into a syringe and subsequently injected into a particle-free sheath stream. Chlorophyll within the phytoplankton is excited by a 635 nm laser and emits fluorescence, which cues a xenon flash lamp and camera trigger. In the IFCB-Sorter, the imaging trigger also begins the sorting sequence: a catcher tube is deployed downstream of the flow cell (moving from the outer particle-free sheath flow into the sample core) to intercept the cell. After entering the catcher tube the cell travels through silicone tubing to a glass capillary tube. Focused on this capillary tube are another 635 nm laser and a microscope objective aimed at a photomultiplier tube (PMT). Chlorophyll is again excited and when the PMT signal exceeds a comparator threshold, a solenoid stops the flow within the capillary tube and a second solenoid shuts off a vacuum line that normally aspirates the stream exiting the capillary to a waste receptacle. Then a solenoid-activated microinjector upstream of the capillary is triggered, which forces the contents of the capillary into a well plate, isolating the cell.

The fluidics system of the IFCB-Sorter differs from that of the standard IFCB in that the flow cell model used is from a Becton Dickinson FACSCalibur™ flow cytometer that is fitted with a deployable catcher tube. The programmable syringe pump (Versapump 6 with a 48,000 step resolution) that is used in the IFCB-Sorter also differs in that it operates a 1 mL syringe (Kloehn, Inc.) as compared to the 5 mL syringe of the IFCB described in Olson and Sosik (2007). The syringe pump is used to sample fluid through a 130 μm Nitex screen, which prevents flow cell clogging. The fluidics of the sorting subsystem includes two solenoid valves, one normally open and one normally closed, for flow control (Bio-chem Fluidics). Once the cell has entered a capillary tube and the cell's emitted fluorescence is detected by a dedicated PMT, the upstream solenoid valve stops flow and vacuum suction ceases. The cell is then stationary in the capillary tube and is flushed out by a microinjector (Bio-chem Fluidics) into a well plate for further analysis.

The optical system of the IFCB-Sorter uses an additional optical system involved in sorting. The optical system involved in sorting is a copy of that in the IFCB. A 635 nm diode laser (SPMT, 635 nm, 12 mW, Power Technologies, Inc.) is focused on the capillary tube of the sorting system by crossed cylindrical lenses. To detect chlorophyll fluorescence a 10X objective is placed at a right angle to the incident laser and focused on the capillary; light collected by this objective is sent to a PMT through a 680 nm bandpass filter to separate the chlorophyll signal from background laser light. The signals and controls of the IFCB-Sorter are similar to that of the original IFCB, again with additional signals and controls related to the sorting process. The additional signals and controls include a catcher tube deployment pulse, which initiates based on the instrument trigger. The catcher tube pulse ramps from 0 to 100 V in 140 μs, plateaus, and then returns to 0 V again in 140 μs. At this time the instrument trigger is inhibited so that cells coming along while the sorted cell is being processed are discarded. When the cell is successfully captured it will then pass through the subsystem laser and emit fluorescence, which is sensed by the second PMT. The signal from the second PMT is fed to a comparator in the PIC, which generates a high going pulse if the PMT signal is larger than a comparator threshold. When the comparator outputs a high-going pulse, a chain of events occurs. First, an upstream solenoid valve (normally open) closes to stop the flow in the capillary tube. Second, a solenoid valve (normally open), which controls removal of waste by a vacuum closes, this stops suction at the end of the capillary tube. Third, the microinjector injects a 20 μL droplet clearing the capillary and sending a droplet containing the cell into the well plate. Fourth, a pulse is sent to the Autoclone well plate system (Coulter Inc.) so that the well plate shifts to a new well. The system then returns to its base state and awaits another trigger event from the IFCB.

Optimization of Timing Parameters—

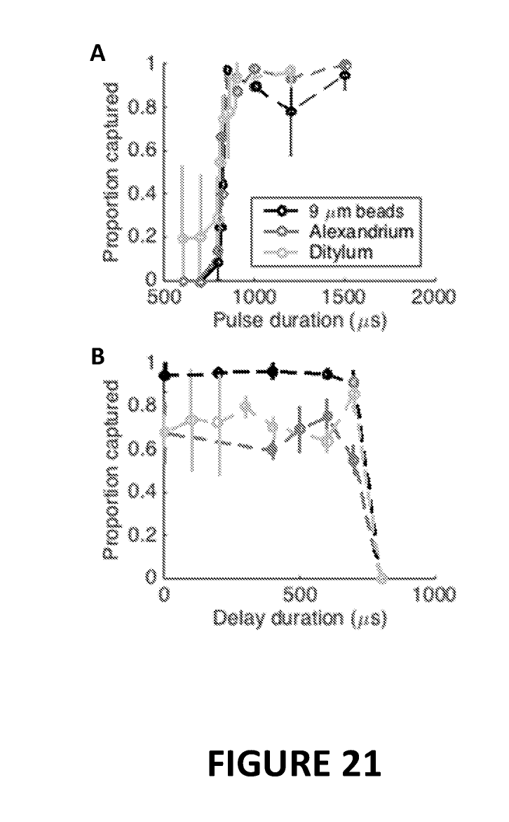

In order to optimize the system and obtain the best capture rate, while minimizing the potential for coincidental capture, we carried out an experiment in which phytoplankton cultures and 9 μm red fluorescent beads (Thermo Fisher Scientific, Inc.) were sorted with varying timing parameter values (FIG. 21). The timing parameters most relevant are the delay from initial IFCB signal until the catcher tube is deployed and the total time that the catcher tube is deployed. The first experiment that we performed involved sorting both phytoplankton cultures and 9 μm red fluorescent beads with no delay between IFCB trigger and catcher tube deployment. By altering the length of catcher tube deployment we were able to bound the time frame in which a capture occurs. The second experiment carried out was holding the sum of the delay between initial trigger and deployment of the catcher tube and the total catcher tube deployment time constant at 900 microseconds. By altering the ratio of delay to deployment duration we were able to obtain operating parameters that maximize capture efficiency and minimize the coincidental capture rate.

Ditylum brightwellii and Alexandrium fundyense used for optimization of timing parameters were kept in f/2 media at 18° C. with a 14:10 Light:Dark cycle. Cells were transferred regularly to ensure they remained in the exponential growth phase.

Quantification of Coincidental Capture—

To identify the rate of coincidental capture we carried out an experiment where a mixture of Dunaliella sp. and Alexandrium fundyense was sorted into a 96-well plate. The IFCB-Sorter was configured so that only the larger A. fundyense would trigger sorting, meaning the Dunaliella sp. were present as a “contaminant” and presence/absence would be indicative of coincidental capture.

To ensure that Dunaliella sp. were viable after sorting, we sorted a culture under the same conditions to be used in determining coincidental capture into a 24-well plate filled with f/2. The result of this control experiment was that 22/24 cells were viable after sorting.

Dunaliella sp. used to quantify coincidental capture rate were grown as described above, whereas the A. fundyense used were kept in f/2 media at 14° C. with a 14:10 Light:Dark cycle.

Quantification of Capture Efficiency for Chain-Forming Cells—

To quantify the capture efficiency for chain-forming cells, a culture of the chain-forming diatom Guinardia delicatula was used as sample. The culture was kept at 11° C. with a 14:10 Light:Dark cycle.

Isolation and Culture of Alexandrium Fundyense—

A. fundyense were cultured as described above. Cells were then sorted into a 96-well plate containing f/2 media. The well plate was then returned to the original incubator. Fluorescence readings were taken on a plate reader to obtain values to construct growth curves.

Isolation and Culture of Cells from Natural Communities—

Cells were sorted from seawater collected from the Iselin Pier at the Woods Hole Oceanographic Institution. The cells were sorted into a 96-well plate containing f/20 media and placed in an incubator at 18° C. with a 14:10 Light:Dark cycle.

Assessment.

The impact of catcher tube deployment timing on capture efficiency—As discussed previously, sending a pulse from the PIC microcontroller deploys the catcher tube. Optimal timing of pulse delivery and the duration of the pulse were determined empirically. FIG. 21A shows the capture efficiency as a function of pulse duration. Based on the results of the first experiment it was determined that most capture occurs between 800 and 900 μs. To refine the timing a second experiment was carried out where the total duration (delay duration and the pulse duration) was fixed at 900 μs. The results are shown in FIG. 21B. From this result a delay time of 700 μs and deployment pulse of 200 μs was chosen as normal operating parameters.

Quantification of Coincidence Rate—

One issue that must be addressed when developing a cell sorter for natural populations is the rate at which you capture more than a single cell. From our sorting parameters and the flow rate of the flow cytometer core, a conservative prediction can be made as to the coincidence rate. Based on a catcher tube deployment time of 700 μs and a flow rate of 0.05 mL/minute an initial volume of −5.8×10⁻⁷ mL is captured by the catcher tube. In order to assess the coincidence rate and compare to the predicted coincidence rate an experiment was designed where a mixture of Dunaliella sp. and Alexandrium fundyense was prepared and sorted. By adjusting the PMT settings, the IFCB-Sorter can be set to trigger only on A. fundyense. Sorting A. fundyense into f/2 media and maintaining the well plate under the same conditions that Dunaliella sp. was initially cultured under, we can check after some time for presence of Dunaliella sp. within the well plate and assess the coincidental capture rate. This experiment was carried out with a background concentration of Dunaliella of 1.3×10⁶ and of 288 sorts performed there were 3 instances of Dunaliella growth. This corresponds to a coincidental capture rate of 0.01 per capture. This is significantly lower than the predicted rate of 0.75 per capture that one would expect from conservative calculations.

Isolation of Cells from the Environment—

To evaluate the effectiveness of the IFCB-Sorter in isolating cells from the environment, seawater was collected for the Iselin Pier at the Woods Hole Oceanographic Institution, Woods Hole, Mass. During the sorting process the PMT high-voltage values were adjusted such that only cells with a large fluorescent signal were selected for sorting. With these PMT parameters most instrument triggers were marine ciliates, large dinoflagellates, and diatoms. The sorted cells were collected in f/20 media and placed in an 18° C. incubator with a 14:10 Light:Dark cycle.

Discussion.

We have introduced here a sorting subsystem for the IFCB platform and addressed the key parameters in construction of such a subsystem. The IFCB-Sorter can effectively sort a variety of phytoplankton, which leads to the possibility of investigating cell-to-cell variability. The critical addition of imaging capabilities to a flow cytometer capable of cell sorting immediately opens up new venues for investigation. Exciting work can now be undertaken using the IFCB-Sorter to probe the relationship between morphology and genotype in marine plankton. One understudied group that could benefit greatly from the application of this technology is marine ciliates. Marine ciliates have long been classified according to morphotype, which requires an expert taxonomist. In recent years it has become apparent that similar morphology does not necessarily coincide with similar genetic similarity. Flow cytometry has supplied crucial information about grazer dynamics in the past. The IFCB-Sorter along with single-cell genomic methods now allows us to investigate these predator-prey interactions in greater detail.

It has also been reported that there can be large genetic dissimilarities in marine planktonic microorganisms resulting in subpopulations forming during blooms and large genetic variability in wild populations of Prochlorococcus. The IFCB-Sorter is an opportunity to attempt to link morphology to this variability and environmental conditions. Additionally the IFCB-Sorter can be used to size-fractionate cells in culture, which can be very valuable for studies in the life cycle of planktonic organisms. These, among others, are exciting avenues to pursue using this instrument.

Comments and Recommendations.

Coincidental capture—The coincidental capture rate was found to be 0.01 per capture. This implies that there should not be much cross contamination of well contents during the sorting process. As this was much lower than the predicted rate, further measurements were made to bound possible coincidence rates. As part of these calculations the mean time to capture was computed from the Alexandrium-Dunaliella coincidental capture experiments. The mean time to capture was 6.3 seconds. With this as a capture time and a measured flow rate through the capillary of 0.029 mL/s, the lower bound on coincidental capture is 8.14×10⁻⁵ per capture. This corresponds to a case where the fluid parcel captured is mixed instantaneously through the entire volume of the capillary and tubing and 20 μL is pulled from that volume. As the observed value is between both this “perfectly mixed” case and the case of plug flow, this implies the sorting system has a flow regime that is intermediate to these ideal cases.

Capture of Chain-Forming Cells—

The sorting system described above is capable of capturing chain-forming cells. It is however with a much lower efficiency than the capture of cells with a simpler morphology. As a proof of concept, the chain-forming diatom Guinardia delicatula was sorted with conservative timing parameters (300 μs delay, 800 μs catcher tube deployment). The result was a capture efficiency of 21.2%±8.1%. It is certainly difficult to capture a chain-forming cell that also has complex morphology, such as setae. An example of a difficult to capture phytoplankton would be long chains of Chaetoceros sp.

Capture of Delicate Cells—

It has long been understood that analysis by flow cytometry can cause physiological damage. While it was initially a concern that sensitive cells would be disrupted during the sorting process, initial trials indicate that the IFCB-Sorter can sort cells traditionally thought to be fragile. Recently cultures of the ciliate Mesodinium rubrum were run through the sorter and active cells were observed within the wells. This demonstrates that sensitive cells will not be affected as much as feared.

Various embodiments and features of the submersible flow imager have been described in detail with particularity. The utilities thereof can be appreciated by those skilled in the art. It should be emphasized that the above-described embodiments of the submersible flow imager merely describe certain examples implementing the submersible flow imager, including the best mode, in order to set forth a clear understanding of the principles of the invention. Numerous changes, variations, and modifications can be made to the embodiments described herein and the underlying concepts, without departing from the spirit and scope of the principles of the invention. All such variations and modifications are intended to be included within the scope of submersible flow imager, as set forth herein. The scope of the submersible flow imager is to be defined by the claims, rather than limited by the foregoing description of various and alternative embodiments. Accordingly, what is desired to be secured by Letters Patent is the invention as described and differentiated in the claims and all equivalents.

For the purpose of understanding the submersible flow imager, references are made in the text to exemplary embodiments of a submersible flow imager, only some of which are described herein. It should be understood that no limitations on the scope of the invention are intended by describing these exemplary embodiments. One of ordinary skill in the art will readily appreciate that alternate but functionally equivalent components, materials, designs, and equipment may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

Reference throughout this specification to features, advantages, or similar language does not imply that all of the features and advantages that may be realized should be or are in any single embodiment. Rather, language referring to the features and advantages is understood to mean that a specific feature, advantage, or characteristic described in connection with an embodiment is included in at least one embodiment. Thus, discussion of the features and advantages, and similar language, throughout this specification may, but do not necessarily, refer to the same embodiment.

Furthermore, the described features, advantages, and characteristics may be combined in any suitable manner in one or more embodiments. One skilled in the relevant art will recognize that the submersible flow imager may be practiced without one or more of the specific features or advantages of a particular embodiment. In other instances, additional features and advantages may be recognized in certain embodiments that may not be present in all embodiments.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment.

It should be understood that the drawings are not necessarily to scale; instead, emphasis has been placed upon illustrating the principles of the invention. In addition, in the embodiments depicted herein, like reference numerals in the various drawings refer to identical or near identical structural elements.

Moreover, the terms “substantially” or “approximately” as used herein may be applied to modify any quantitative representation that could permissibly vary without resulting in a change to the basic function to which it is related.

1. A device for processing particles within liquid suspensions comprising:
a flow system, through which a liquid suspension, comprising suspended particles, travels;
a detection system, adapted to detect one or more aspects of the suspended particles passing through the detection system;
an electronics system, electronically connected to said flow system and said detection system; and
a watertight housing;
wherein the liquid suspension is imbibed into the flow system and transferred to the detection system for particle processing, and the flow system, the detection system, and the electronics system are disposed within the housing; and wherein the device comprises a modular device and a low relief configuration.

2. The device of claim 1, further comprising a bubble minimization assembly.

3. The device of claim 2, wherein the bubble minimization assembly comprises:
one or more tubes; and
a valve;
wherein said one or more tubes syphon a bubble-containing fluid and said bubble-containing fluid is expelled from said device, through said valve, and into an outside environment.

4. The device of claim 1, wherein the flow system further comprises:
a sample intake means, configured to imbibe said liquid suspension from a surrounding environment;
a pumping means, providing motive force to the imbibed liquid;
a delivery means, capable of transferring said liquid suspension from the sample intake means to a detection system; and
at least one valve.

5. The device of claim 1, wherein the detection system further comprises:
a detection path, coupled to the pumping means through a delivery means;
a detection interface subsystem, comprising a flow cell, a core, and a sheath; and
an optical system.

6. The device of claim 1, further comprising an acoustic focusing element for the in-line concentration of samples, comprising said liquid suspensions, in the flow system.

7. The device of claim 1, wherein the electronics system comprises:
a controller;
a data acquisition device;
a power source; and
a data processor;
wherein said electronics system provides control for both movement of said liquid suspension, within said flow path, the detection of said particles, and data storage and transmission.

8. The device of claim 1, further comprising a particle suspension means comprising a syringe pump to impart turbulence to prevent said particles from settling.

9. The device of claim 1, wherein the device height of said device is constrained by the height of the detection path.

10. The device of claim 1, wherein the height of said device is less than approximately 48 cm.

11. The device of claim 1, wherein said device further comprises an attachment means for attaching said device to a vehicle.

12. The device of claim 1, wherein said particle processing is selected from including imaging said particle, staining said particle, sorting said particle, observing said particle, counting said particle, isolating said particle, and any combination thereof.

13. The device of claim 1, further comprising an automated staining system, said automated staining system comprising:
a microinjector;
a stain to dye said particles for imaging;
a stain reservoir for storage of said stain; and
a mixing chamber capable of receiving said stain from said stain reservoir via a delivery means;
wherein a sample intake means intakes a sample, which comprises said liquid suspension, and said microinjector injects said stain from said stain reservoir to said mixing chamber, then a pumping means propels said sample into said mixing chamber, where said sample mixes and incubates with said stain, then said pumping means propels said sample back through said delivery means and into a sample syringe, and then said flow system delivers said sample into a flow cell for analysis.

14. The device of claim 1, further comprising an automated sorting system, said automated sorting system comprising:
a catcher tube, located to receive one or more said particles from a flow cell;
a piezo element, which functions to position said catcher tube to receive said one or more particles from said flow cell;
a valve, capable of shutting off flow of said flow cell to allow said one or more particles to be analyzed; and
a laser and detector assembly, to verify that said one or more particles were captured and which enables the release of a drop of a sheath fluid said one or more particles, to be stored and preserved.

15. A system comprising:
a vehicle, capable of receiving a device comprising a low relief configuration, said vehicle being operational in water;
a flow system, through which liquid suspensions, comprising particles, travel;
a detection system, adapted to detect one or more aspects of said particles flowing through said detection system;
an electronics system, electronically connected to said flow system and said detection system; and
a watertight housing;
wherein said liquid suspension is imbibed into said flow system and transferred to said detection system for particle processing, said flow system, said detection system, and said electronics system are located within said watertight housing, and said watertight housing is removably attached to the vehicle.

16. The system of claim 15, further comprising a bubble minimization assembly capable of removing bubbles from said flow system when said device is substantially horizontal.

17. The system of claim 16, wherein the bubble minimization assembly comprises:
one or more tubes; and
a valve
wherein said one or more tubes syphon said bubbles from a bubble-containing fluid located in said flow system and said valve expels said bubble-containing fluid, from said device to the outside environment.

18. The system of claim 15, wherein said flow system further comprises:
a sample intake means, configured to imbibe said liquid suspension from a surrounding environment;
a pumping means, proving motive force to the said liquid suspension;
a delivery means, capable of transferring said liquid suspension from said sample intake means to said detection system; and
at least one valve.

19. The system of claim 15, wherein said detection system further comprises:
a detection path, coupled to a pumping means through a delivery means;
a detection interface subsystem, comprising a flow cell, a core, and a sheath through which a sheath fluid flows; and
an optical system.

20. The system of claim 15 wherein said electronics system comprises:
a controller;
a data acquisition device;
a power source; and
a data processor;
wherein said electronics system provides control for movement of said liquid suspension within said flow path, the detection of said particles, and data storage and transmission.

21. The system of claim 15, wherein the height of said device is dictated by the height of a detection path.

22. The system of claim 15, wherein said device comprises a height of less than 48 CM.

23. The system of claim 15, wherein said device further comprises an automated staining system, said automated staining system comprising:
a microinjector;
a stain to dye said particles for imaging;
a stain reservoir for storage of said stain; and
a mixing chamber capable of receiving said stain from said stain reservoir via a delivery means;
wherein a sample intake means intakes a sample, which comprises said liquid suspension, said microinjector adds said stain from said stain reservoir to said mixing chamber, and a pumping means propels said sample into said mixing chamber, where said sample mixes and incubates with said stain, then said pumping means propels said sample back through said delivery means and into a sample syringe, and then said flow system delivers said sample into a flow cell for analysis.

24. The system of claim 15, wherein said device further comprises an automated sorting system, said automated sorting system comprising:
a catcher tube, located to receive one or more said particles from a flow cell;
a piezo element, that functions to position said catcher tube to receive said particle from said flow cell;
a valve, capable of shutting off flow of said flow cell to allow said particles to be analyzed; and
a laser and detector assembly, said laser and detector assembly verifies that said particle was captured and enables the timed release of a drop a sheath fluid, comprising said particle, to be preserved.
